(19) United States
(12) Patent Application Publication
(10) Pub. No.: US 2014/0235458 A1
(43) Pub. Date: Aug. 21, 2014

(54) METHODS AND TOOLS FOR THE DIAGNOSIS AND PROGNOSIS OF UROGENITAL CANCERS

(71) Applicant: Cancer Genetics, Inc., Rutherford, NJ (US)

(72) Inventors: Jane C. Houldsworth, Franklin Lakes, NJ (US); Raju S.K. Chaganti, Hillsdale, NJ (US)

(73) Assignee: Cancer Genetics, Inc., Rutherford, NJ (US)

(21) Appl. No.: 14/180,814

(22) Filed: Feb. 14, 2014

Related U.S. Application Data
(60) Provisional application No. 61/765,678, filed on Feb. 15, 2013.

(51) Int. Cl. C12Q 1/68

(52) U.S. Cl. CPC C12Q 1/6886
USPC 506/2; 435/6.11; 506/9; 506/16

(57) ABSTRACT

The present invention provides a microarray useful as a tool in the diagnosis and/or prognosis of certain types of cancers, particularly urogenital cancers. The microarray can include a plurality of genomic regions represented thereon, the genomic regions corresponding to regions wherein alterations, such as copy number aberrations, at such locations correlate to specific, identifiable cancers, particularly prostate, renal, or bladder tumors. The invention further provides methods of diagnosing certain types of cancers, particularly urogenital cancers, more particularly renal cortical cancers. The methods can comprise analyzing genetic material from a human individual to determine the presence or presence of certain aberrations and using a decision tree to classify the subtype of renal cortical neoplasm present in the sample.

### CROSS-REFERENCE TO RELATED APPLICATION

This application claims the benefit of U.S. Provisional Application No. 61,765,678, filed Feb. 15, 2013, which is hereby incorporated herein in its entirety by reference.

### FIELD OF THE INVENTION

The present invention relates to cancer and in particular to urogenital cancers. In particular, the present invention relates to methods and tools for the diagnosis and prognosis of prostate, kidney, and bladder cancers. The invention also provides methods for the diagnosis and prognosis of such malignancies using alternative platforms or technologies, preferentially with minimal invasiveness.

### BACKGROUND OF THE INVENTION

The reproductive organs (prostate, testis, penis, cervix, uterine, ovary, vulva, and vagina) and the urinary system (two kidneys, bladder, combined with the urine transporting ureters and urethra) are commonly grouped together as the genitourinary system due to their physiological and developmental proximity and sharing of the excretory pathways (Abrahams (2002) 256 p.). In 2011, cancers of the genitourinary system account for close to 30% of all estimated new cancer cases in the U.S. and the three most prevalent types are prostate, bladder, and kidney cancers (51%, 15%, and 13% of all estimated genitourinary cancer cases respectively, see Table 1) (Jemal et al. (2010) CA Cancer J Clin 60:277-300).

TABLE 1
Estimated Frequencies of Genitourinary Cancers in the U.S. in 2011

| | Estimated New | Percentage of Total |
|---|---|---|
| Cancer Type | Cases in 2011 | Cancer Cases in 2011 |
| Prostate | 240,890 | 51% |
| Urinary Bladder | 69,250 | 15% |
| Kidney (including renal pelvis) | 60,290 | 13% |
| Uterine corpus | 46,470 | 10% |
| Ovary | 21,990 | 5% |
| Uterine cervix | 12,200 | 3% |
| Testis | 8,480 | 2% |
| Vulva | 4,340 | 1% |
| Vagina and other genital, female | 2,570 | ~0.4% |
| Ureter and other uniary organs | 2,730 | ~0.4% |
| Penis and other genital, male | 1,360 | ~0.2% |

The estimated deaths caused by the three types of cancer in 2011 are 33,720, 14,990, and 13,120 respectively, comprising ˜11% of all cancer-related deaths (Jemal et al. (2010) CA Cancer J Clin 60:277-300). Thus, although they are generally characterized by early stage detections and encouraging five-year survival rates (Hayat et al. (2007) Oncologist 12:20-37), these three types of genitourinary cancers still represent major health risk and substantial medical cost burden to the public with their high rates of incidence. Thus, developing sophisticated, state-of-the-art molecular assays that enable more accurate diagnosis and/or prognosis of these cancers will not only benefit the patients by offering more appropriate treatments but also effectively reduce the unnecessary medical cost associated with surgery, long-term follow-up surveillance, or adjuvant therapy after the treatment.

In United States, prostate cancer is the most commonly detected cancer (Jemal et al. (2010) CA Cancer J Clin 60:277-300). It is also the third leading cause of cancer-related death in men (Jemal et al. (2010) CA Cancer J Clin 60:277-300). Prostate tumors exhibit considerable heterogeneity as demonstrated by a tremendous variability in the rate of tumor progression in the patient population and greater rates of incidence and mortality in the African American males comparing to other ethnic populations (Hayat et al. (2007) Oncologist 12:20-37). Approximately 95% of prostate cancers are adenocarcinomas (Bostwick (1989) CA Cancer J Clin 39:376-93) in which deregulation of many major biochemical pathways have been identified. After the initial tumor formation, further alterations in genes such as TP53, BCL2, PTEN, and RB that govern cell cycle regulation or apoptosis can lead to tumor progression and metastasis and many therapies have been designed to target these pathways (Lee et al. (2008) Cell Cycle 7:1745-62; Pommery et al. (2005) Mini Rev Med Chem 5:1125-32). In the last 20 years, the increasing adaptation of the prostate-specific-antigen (PSA) test using PSA level in blood samples as a biomarker for prostate tumor has dramatically increased the number of new prostate cancer cases detected at early stages (Rao et al. (2008) BJU Int 101:5-10). However, for a disease with a potentially long progression time, the challenge arises in determining the most appropriate and cost-effective treatment for each patient after the initial diagnosis. For example, simple active surveillance may be recommended in place of surgery or other more aggressive treatments if certain criteria are met (NCCN Clinical Practice Guidelines in Oncology-Prostate Cancer, (2010) National Comprehensive Cancer Network, Fort Washington, Pa.) in order to prevent overtreatment which significantly affect morbidity in an aging patient population (Daskivich et al. (2011) Cancer 117:2058-66). Thus, in prostate cancer, risk-adapted patient management is highly desired. Patient stratification in cancer generally involves estimating the potential risk of a detected tumor based on some pre-established factors that are known to affect the eventual disease outcome and then recommending a particular treatment accordingly. Patient stratification in prostate cancer is commonly achieved by using nomograms developed based on a combination of Gleason score, PSA level, and tumor staging (NCCN Clinical Practice Guidelines in Oncology-Prostate Cancer, (2010) National Comprehensive Cancer Network, Fort Washington, Pa.). Furthermore, the initial recommended treatment method is also influenced by patient age, potential therapy side effects, and patient preference (NCCN Clinical Practice Guidelines in Oncology-Prostate Cancer, (2010) National Comprehensive Cancer Network, Fort Washington, Pa.). Although the current nomograms have improved prostate cancer patient management (Zaytoun et al. (2011) Urology; Paris et al. (2010) Clin Cancer Res 16:195-202; Korets et al. (2011) BJU Int 108:56-60), additional prognostication for biochemical recurrence or metastasis is still needed.

Genomic aberrations are the hallmark of cancer cells (Colnaghi et al. (2011) Semin Cell Dev Biol 22:875-85) and many have the potential to offer disease-specific diagnostic and/or prognostic. The current nomograms used for prostate cancer patient management do not incorporate genomic markers despite the fact that genomic instabilities have been studied extensively in prostate tumor samples over the years (Cher et al. (1996) Cancer Res 56:3091-102; Nupponen et al. (1998) Am J Pathol 153:141-8; Sato et al. (1999) J Natl Cancer Inst 91:1574-80; Fu et al. (2000) Urology 56:880-5; Chu et al. (2001) Cancer Genet Cytogenet 127:161-7; van Dekken et al. (2004) Genes Chromosomes Cancer 39:249-56; Yano et al. (2004) Cancer Genet Cytogenet 150:122-7; Kindich et al. (2006) Eur Urol 49:169-75; Liu et al. (2006) Genes Chromosomes Cancer 45:1018-32; Hughes et al. (2006) BMC Genomics 7:65; Saramaki et al. (2006) Int J Cancer 119:1322-9; Paris et al. (2006) Neoplasia 8:1083-9; Kim et al. (2007) Cancer Res 67:8229-39; Lapointe et al. (2007) Cancer Res 67:8504-10; Kobayashi et al. (2008) Prostate 68:1715-24; Fukasawa et al. (2008) Prostate Cancer Prostatic Dis 11:303-10; Holcomb et al. (2009) Cancer Res 69:7793-802; Demichelis et al. (2009) Genes Chromosomes Cancer 48:366-80; Liu et al. (2009) Nat Med 15:559-65; Castro et al. (2009) Neoplasia 11:305-12; Mao et al. (2010) Cancer Res 70:5207-12; El Gedaily et al. (2001) Prostate 46:184-90; Watson et al. (2004) Oncogene 23:3487-94; Bock et al. (2009) Hum Genet 126:637-42; Chen et al. (2010) Prostate Cancer Prostatic Dis 13:238-43). These experiments, often with small sample sets, were performed with varied methodologies and platforms. Thus, many of the frequently altered genomic aberrations in prostate cancer and their potential consequences associated with clinical outcomes are only somewhat consistent across different studies. In the western population, an interstitial deletion at chromosome 21q22.2-22.3.3 that caused the fusion between the TMPRSS2 gene promoter which is regulated by androgenic hormones and the ERG gene, an oncogene that is a member of the of the erythroblast transformation-specific (ETS) family of transcriptions factors, occurs in approximately 50% of all prostate cancers (Demichelis et al. (2009) Genes Chromosomes Cancer 48:366-80; Lapointe et al. (2007) Cancer Res 67:8504-10; Liu et al. (2006) Genes Chromosomes Cancer 45:1018-32; Kim et al. (2007) Cancer Res 67:8229-39; Holcomb et al. (2009) Cancer Res 69:7793-802). Putting expression of ERG under androgen regulation results in its over-expression in prostate cancer cells. Occasionally, fusions involving other members of the ETS family of transcription factors such as ETV1, ETV4 and ETV5 are also found with diverse 5′ partners resulting in the up-regulation of these genes during androgen response in most of the cases (Clark et al. (2009) Nat Rev Urol 6:429-39). Over 40% of prostate tumors have altered PTEN/Akt expression (Majumder et al. (2005) Oncogene 24:7465-74) which regulates a broad spectrum of cellular functions (Georgescu (2010) Genes Cancer 1:1170-7) including both cell cycle and apoptosis. The gene encoding PTEN is localized on chromosome 10q23 and genomic aberrations are often found in this region in prostate tumors (Liu et al. (2006) Genes Chromosomes Cancer 45:1018-32; Lapointe et al. (2007) Cancer Res 67:8504-10; Holcomb et al. (2009) Cancer Res 69:7793-802; Kim et al. (2007) Cancer Res 67:8229-39). In prostate tumors, the majority of PTEN disruptions are deletions while point mutations occur as minor events (de Muga et al. (2010) Mod Pathol 23:703-12). The presence of the TMPRSS2:ERG fusion and the loss of the PTEN protein are associated with poorer disease outcome (Yoshimoto et al. (2008) Mod Pathol 21:1451-60; Sircar et al. (2009) J Pathol 218:505-13). Interestingly, these two often detected genomic aberrations are found at much lower frequency in the Chinese population suggesting alternative pathogenetic pathways (Mao et al. (2010) Cancer Res 70:5207-12). Copy number gain of the 8q24 region which harbors the MYC gene is also detected frequently in prostate tumor genomes and has been implicated in tumor grade and disease progression and outcome (Saramaki et al. (2006) Int J Cancer 119:1322-9; Chen et al. (2010) Prostate Cancer Prostatic Dis 13:238-43; Sato et al. (1999) J Natl Cancer Inst 91:1574-80). In 2010, a comprehensive genome-wide study suggested that patterns of genomic copy number aberrations or alterations (CNA) being either loss or gain of genomic material, can be used to stratify patients into different outcome groups (Taylor et al. (2010) Cancer Cell 18:11-22). This finding lends support for developing diagnostic/prognostic assays based on a limited number of specific genomic aberrations that are associated with disease progression and outcome.

Kidney cancer represents approximately 4% of all cancers in the US and it is the 8th most prevalent cancer overall (Jemal et al. (2010) CA Cancer J Clin 60:277-300). Kidney cancer is noticeably a disease of the older population, where the median age of diagnosis is 64 years (Hayat et al. (2007) Oncologist 12:20-37). The majority of kidney tumors arises from the renal epithelium within the kidney and they can be further sub-divided based on their histologic features and cytogenetic makeups (Lopez-Beltran et al. (2006) Eur Urol 49:798-805). The malignant renal cell carcinoma (RCC) originated from renal epithelial cells is not a single entity but comprises of multiple subtypes with the three most prevalent ones being clear cell RCC, papillary RCC, and chromophobe RCC. Greater than 85-90% of kidney malignancies found are RCCs and the disease progression is often aggressive (Hayat et al. (2007) Oncologist 12:20-37). On the other hand, renal oncocytoma (RO), a frequently detected renal epithelial neoplasm, is benign.

Kidney cancer usually is first noticed as a suspicious mass involving the kidney that was found using radiographic analysis such as CT scans or even by ultrasound. In some cases, the scan was performed because the patients had symptoms caused by the mass, or performed for some other condition that incidentally lead to the detection of the mass. Now, 60-70% of patients with RCC are asymptomatic at the time of diagnosis. The widespread of modern imaging techniques has led to an increased detection of incidental and smaller kidney masses, leading to the difficult question of when and to what extent the physician should intervene. Most suspicious solid kidney masses are removed by radical nephrectomy (NCCN Clinical Practice Guidelines in Oncology-Prostate Cancer, (2010) National Comprehensive Cancer Network, Fort Washington, Pa.). In order to avoid unnecessary nephrectomy, image-guided tumor biopsy can be obtained for further analysis before a surgical procedure is performed. Indeed, the importance of such diagnostic procedures is highlighted in recent studies shown close to a third of small renal masses biopsied were benign, providing support for non-surgical treatment in these individuals (Tuncali et al. (2004) AJR Am J Roentgenol 183:575-82). There is a growing body of evidence to suggest that this procedure will become part of the routine care of the increasing number of patients identified to have a renal mass and/or kidney cancer. Despite the recognized current limitations of the procedure, kidney biopsy has already been included as an option in the NCCN Guidelines when warranted for diagnostic purposes (NCCN Clinical Practice Guidelines in Oncology-Prostate Cancer, (2010) National Comprehensive Cancer Network, Fort Washington, Pa.).

Although the procedure of kidney biopsy is associated with little complications and demonstrable clinical significance, the challenge lies in the accurate diagnosis to distinguish between the malignant tumors (mostly RCCs) from the benign ones, most frequently the renal oncocytoma, using such minimal amount of material. Often, small renal mass biopsies could not be diagnosed due to either the failure to obtain biopsy tissue of adequate quantity and/or quality for pathological examination or the inability to distinguish RCC subtypes pathologically. In fact, a significant percentage (around 7%) of renal RCCs are “unclassified” due to unusual morphological features (Eble et al., eds. (2004) Pathology and Genetics, Tumors of the Urinary System and Male Genital Organs, World Health Organization, International Agency for Research on Cancer, Lyon, France). A highly sensitive, independent molecular assay using materials extracted from the biopsied tissues for the identification and classification of renal masses will enhance the diagnostic power and accuracy of such procedure. For each subtype of the renal tumors, a specific pattern of genomic aberrations is often observed and potentially such patterns can be used as classifiers. Indeed, such a possibility has been explored at the research level (Vieira et al. (2010) Genes Chromosomes Cancer 49:935-47; Mai et al. (2010) Virchows Arch 456:77-84; Haudebourg et al. (2010) Virchows Arch 457:397-404; Hagenkord et al. (2008) Diagn Pathol 3:44; Hagenkord et al. (2011) Cancer Genet 204:285-97; Yusenko et al. (2009) BMC Cancer 9:152) but due to sample size limitations and differences in cohort population, there is no standard consensus in the diagnostic scheme that has been rigorously validated and translated to clinical practice.

Genomic aberrations associated with different clinical features of renal cancer have also been extensively studied at the discovery phase (i.e. small number of samples with non-standardized platforms) (Monzon et al. (2008) Mod Pathol 21:599-608; Pei et al. (2010) Genes Chromosomes Cancer 49:610-9; Klatte et al. (2009) J Clin Oncol 27:746-53; Klatte et al. (2009) Clin Cancer Res 15:1162-9; Szponar et al. (2009) Int J Cancer 124:2071-6; Gunawan et al. (2001) Cancer Res 61:7731-8; Jiang et al. (1998) J Pathol 185:382-8; Yao et al. (2002) J Natl Cancer Inst 94:1569-75; Beroukhim et al. (2009) Cancer Res 69:4674-81; Toma et al. (2008) Neoplasia 10:634-42; Brunelli et al. (2008) Mod Pathol 21:1-6; Pavlovich et al. (2003) Genes Chromosomes Cancer 37:252-60; Arai et al. (2008) Clin Cancer Res 14:5531-9; Bissig et al. (1999) Am J Pathol 155:267-74; Chen et al. (2009) Int J Cancer 125:2342-8; Wilhelm et al. (2002) Cancer Res 62:957-60; Jiang et al. (1998) Am J Pathol 153:1467-73; Yusenko et al. (2009) BMC Cancer 9:152; Morita et al. (1991) Cancer Res 51:5817-20; Presti et al. (1998) J Urol 160:1557-61; Strefford et al. (2005) Cancer Genet Cytogenet 159:1-9; Gronwald et al. (1997) Cancer Res 57:481-7; Yoshimoto et al. (2007) J Pathol 213:392-401; Moch et al. (1996) Cancer Res 56:27-30; Nagao et al. (2005) Cancer Genet Cytogenet 160:43-8; Brannon et al. (2010) Genes Cancer 1:152-63; Reutzel et al. (2001) Cytogenet Cell Genet 93:221-7). Most of the studies have been focused on clear cell RCC (ccRCC) since it is the most prevalent subtype of renal cell carcinoma. To date, several of the genomic aberrations have been implicated in predicting ccRCC progression into the metastatic state and the overall survival rate. Loss of 3p including the 3p25 region that harbors the Von Hippel-Lindau (VHL) disease gene, is the most commonly found copy number change in ccRCC. The VHL gene is altered frequently in clear cell RCC (Shuin et al. (1994) Cancer Res 54:2852-5; Gnarra et al. (1994) Nat Genet 7:85-90). The loss of 3p has been found to associate with lower stage, grade, and lower chance of distant metastasis and favorable prognosis (Bissig et al. (1999) Am J Pathol 155:267-74; Gronwald et al. (1997) Cancer Res 57:481-7; Klatte et al. (2009) J Clin Oncol 27:746-53; Yao et al. (2002) J Natl Cancer Inst 94:1569-75). Similarly, gain of 5q predicts favorable prognosis and lower grade (Reutzel et al. (2001) Cytogenet Cell Genet 93:221-7; Gunawan et al. (2001) Cancer Res 61:7731-8). On the other hand, loss of 9p or loss of 14q is associated with poor prognosis, higher tumor grade and increased chance of distant metastasis (Chen et al. (2009) Int J Cancer 125:2342-8; Arai et al. (2008) Clin Cancer Res 14:5531-9; Klatte et al. (2009) J Clin Oncol 27:746-53; Yoshimoto et al. (2007) J Pathol 213:392-401). A recent study has also suggested that the loss of 4q has a role in tumor metastasis (Lopez-Lago et al. (2010) Cancer Res 70:9682-92) which is supported by previous publications (Arai et al. (2008) Clin Cancer Res 14:5531-9; Reutzel et al. (2001) Cytogenet Cell Genet 93:221-7; Yoshimoto et al. (2007) J Pathol 213:392-401).

Urinary bladder cancer is a common malignant disease that has the third highest estimated rate of incidence in the United States in 2010 (Jemal et al. (2010) CA Cancer J Clin 60:277-300). Bladder tumors have two basic forms: the localized, low-grade papillary exophytic tumors and the solid, invasive cancers with higher grades of cellular dedifferentiation (NCCN Clinical Practice Guidelines in Oncology-Prostate Cancer, (2010) National Comprehensive Cancer Network, Fort Washington, Pa.). Carcinoma in situ (CIS), non-invasive but high grade intra-urothelial neoplasia often found concomitant with invasive cancers, is believed to be the precursor of invasive cancers (Zieger et al. (2011) Scand J Urol Nephrol). At the molecular level, alterations in different biochemical pathways have also been found for the two different bladder cancer forms. The papillary tumors often contain mutations in FGFR3 gene while the invasive cancers are marked by mutations in the TP53 gene and increased genetic instability (Zieger et al. (2009) Int J Cancer 125:2095-103).

Approximately, 70-75% of newly diagnosed bladder tumors are locally confined, low-grade, papillary tumors for which the standard recommended treatment is TURBT (transurethral resection of bladder tumor) (NCCN Clinical Practice Guidelines in Oncology-Prostate Cancer, (2010) National Comprehensive Cancer Network, Fort Washington, Pa.); Sexton et al. (2010) Cancer Control 17:256-68). However, these non-invasive tumors have a high rate of reoccurrence after the initial removal and 10-70% of patients will experience either a reoccurrence or a new occurrence of urothelial carcinoma within 5 years (NCCN Clinical Practice Guidelines in Oncology-Prostate Cancer, (2010) National Comprehensive Cancer Network, Fort Washington, Pa.). In addition, a significant percentage (10-20%) of the recurrent tumors progress to a higher grade and/or higher stage [85]. The probability of progression varies with initial stage, grade, and size of the tumor and currently, there is no reliable biomarker that can distinguish tumors with the more aggressive characteristics from the ones that have low reoccurring potential.

After the initial resection, intravescial chemotherapy using either Bacillus Calmette-Guérin (BCG) or mitomycin C is often used as an adjuvant therapy. However, given the median age at diagnosis for cancer of the urinary bladder is around 73 years of age (Hayat et al. (2007) Oncologist 12:20-37), any additional therapy is considered carefully. In many cases, intravescial therapy may be overly used if the prognosis is good and the chance of reoccurrence is low [85]. Therefore, after TURBT, a sensitive and consistent assay which will independently evaluate the chance for recurrence and tumor progression will be valuable in helping the physicians in deciding whether to order additional adjuvant treatments or adjust the amount of treatment accordingly. Chromosomal imbalances have been reported to be associated with tumor progression, reoccurrence, and staging in the early stage bladder carcinomas (Chan et al. (2009) Int J Oncol 34:963-70; Nord et al. (2010) Int J Cancer 126:1390-402; Presti et al. (1991) Cancer Res 51:5405-9; Yamamoto et al. (2007) Oncology 72:132-8; Kawanishi et al. (2007) Br J Cancer 97:260-6; Kallioniemi et al. (1995) Genes Chromosomes Cancer 12:213-9; Richter et al. (1997) Cancer Res 57:2860-4; Wagner et al. (1997) Am J Pathol 151:753-9; Kagan et al. (1998) Oncogene 16:909-13; Hovey et al. (1998) Cancer Res 58:3555-60; Zhao et al. (1999) Cancer Res 59:4658-61; Qin et al. (2006) Cancer Lett 238:230-9; Prat et al. (2010) Urology 75:347-55; Williams et al. (2010) Genes Chromosomes Cancer 49:642-59; Hurst et al. (2004) Oncogene 23:2250-63; Lindgren et al. (2010) Cancer Res 70:3463-72; Bruch et al. (1998) Genes Chromosomes Cancer 23:167-74; Eguchi et al. (2010) Cancer Genet Cytogenet 200:16-22). Furthermore, specific genetic alterations in the muscle-invasive, metastatic bladder tumors are also documented (Pollard et al. (2010) Expert Rev Mol Med 12:e10; Heidenblad et al. (2008) BMC Med Genomics 1:3). One specific genomic aberration that has been reported by multiple publications is the loss of chromosome 8p that is associated with tumor progression and recurrence (Eguchi et al. (2010) Cancer Genet Cytogenet 200:16-22; Bruch et al. (1998) Genes Chromosomes Cancer 23:167-74; Wagner et al. (1997) Am J Pathol 151:753-9).

From the studies cited above, it is clear that genomic instability has been studied in research settings in order to identify potential candidate genes and biochemical pathways involved in tumor biology (Colnaghi et al. (2011) Semin Cell Dev Biol). Those were mostly recognized initially through traditional cytogenetic studies and often now are assessed by molecular cytogenetic techniques such as fluorescence in situ hybridization (FISH) or by molecular genetic techniques such as PCR. Over the years, however, much research effort has been expended in order to identify robust oncological biomarkers at the DNA, RNA, and protein levels. It was not until genome scanning technologies such as comparative genomic hybridization (CGH), were introduced and became reliable, did the role of genomic gain and loss in tumor formation and progression become apparent and reproducible in high resolution (Davies et al. (2005) Chromosome Res 13:237-48). These technologies have evolved for the examination of chromosome abnormalities with differing technical advantages/disadvantages. Examples are shown below in Table 2.

TABLE 2
Common Technologies for Genomic Aberration Detection

| | | | | | Multiplex |
|---|---|---|---|---|---|
| Technique | Coverage | Resolution | | Aberrations Detected | Capability |
| Karyotyping | Whole genome | >10 | Mbp | Rearrangement (balanced, | |
| | | | | unbalanced), gain, loss | |
| SKY | Whole genome | >2 | Mbp | Rearrangement (balanced, | |
| | | | | unbalanced), gain, loss | |
| Chromosomal-CGH | Whole genome | >2 | Mbp | Gain, loss | |
| FISH | Probe-specific | >200 | kbp | Rearrangement (balanced, | ✓✓ |
| | | | | unbalanced), gain, loss | |
| Array-CGH | Whole genome/Focused | 5-100 | kbp | Rearrangement (unbalanced), | ✓✓✓ |
| | | | | gain, loss | |
| SNP-Array | Whole genome/Focused | 5 | kbp | Gain, loss, uniparental disomy, | ✓✓✓ |
| | | | | mutation | |
| PCR | Gene-specific | <10 | kbp | Rearrangement (balanced, | ✓✓ |
| | | | | unbalanced), gain, loss, mutation | |
| Southern Blotting | Gene-specific | <20 | kbp | Rearrangement (balanced, | ✓ |
| | | | | unbalanced), gain, loss | |
| Massively Parallel | Whole genome/Focused | 1 | bp | Rearrangement (balanced | ✓✓✓ |
| Sequencing | | | | unbalanced), gain, loss, mutation | |

The above technologies provide variable degrees of usefulness and require the following specific considerations: karyotyping and FISH are labor-intensive with little automation; SKY, chromosomal-CGH, FISH, array-CGH, SNP-array, and Massively Parallel Sequencing require costly reagents/equipment; karyotyping requires growth of cells while chromosomal-CGH, array-CGH, SNP-array, PCR, Southern blotting and Massively Parallel Sequencing only require DNA, chromosomal-CGH, array-CGH, SNP-array and Massively Parallel Sequencing require algorithmic analysis; PCR, FISH, Southern blotting offer greater sensitivity while array-CGH, SNP-Array, and Massively Parallel Sequencing provide much better scanning power at the genomic level. In a clinical diagnostic setting, karyotyping, FISH, PCR, and to a much reduced extent Southern blotting, have been the preferred technologies, and the American College of Medical Genetics (ACMG) has established Standards and Guidelines for these technologies. Standards and Guidelines (Kearney et al. (2011) Genet Med 13:676-79) have been suggested for the performance of array-CGH as a replacement for (or as an adjunct to) standard cytogenetic techniques (e.g., karyotyping, FISH) as commercially available FDA-approved devices, as commercially available Investigational Use Only (IUO) devices requiring validation, or as “home-brew” or in-house developed and validated devices; however, they have not yet been adopted. To date, array-CGH has been utilized primarily as “home-brew” assays.

With increasing resolving power afforded by oligonucleotide arrays, smaller recurrent gains and losses have been identified and common regions of genomic gain/loss have been narrowed. Few alterations have been reported to be associated with the disease or a biologic or clinical feature of the disease. Numerous studies in the past decade have been published utilizing array-CGH for molecular characterization of prostate cancers, renal cancers, and bladder cancers. Some of these analyses are done in a limited set of patient samples reporting the most significant aberrations found in general for a specific type or subtype of genitourinary cancer. Other studies are carried out with larger group of patients and reported aberrations that are associated with a specific clinical outcome of the disease. With respect to prostate, renal, and bladder malignancies, the role of genomic gain and loss is still in the discovery phase and the full potential of genomic gain/loss as diagnosticators and prognosticators has yet to be explored and exploited in a clinical setting.

### BRIEF SUMMARY OF THE INVENTION

The present invention provides methods and tools for the assessment of genomic aberrations in the diagnosis and prognosis of cancer and precancer, particularly prostate, renal, and bladder malignancies. In one embodiment, the invention provides methods that can involve the use a genome scanning technology, such as, for example, array comparative genomic hybridization (array-CGH), as a clinical tool for the diagnosis and prognosis of prostate, renal, and bladder cancers. The invention further provides additional methods, platforms, specimen cohort sizes, and treatment modalities that, when used for prostate, renal, and bladder cancers described herein, can be useful in diagnosis and prognosis of these cancers in a sample.

In one aspect, the present invention provides an oligonucleotide-based urogenital cancer microarray, which is also known as the UroGenRA® urogenital cancer array, for predicting recurrence of prostate cancer after surgery. In another aspect, the invention provides a microarray for determining if low risk insignificant prostate cancers are under-staged or under-graded and have an unfavorable prognosis. The invention further provides a microarray for determining response of intermediate risk prostate cancers to radiation treatment. In another aspect, the invention can diagnose, classify, and distinguish between the four main subtypes of renal cell carcinoma (RCC) in needle, core, and resected biopsy material. For each malignant subtype of renal cancer, the invention provides a microarray for prediction of response treatment and overall outcome. In the case of bladder cancer, the invention can determine the metastatic potential of a biopsy or resected cancer to indicate the need for chemotherapy. In certain embodiments a microarray according to the invention can comprise a substrate with a plurality of nucleic acid molecules corresponding to distinct genomic regions arrayed thereon. Preferably, the nucleic acid molecules corresponding to the distinct genomic regions individually can be capable of hybridizing to material present in the sample. Moreover, the genomic regions arrayed on the substrate can be regions wherein an alteration therein is correlated to cancer type and subtype, and/or clinical outcome in prostate, renal, and bladder cancers.

In addition, the invention also provides methods for determining the type or predicting the disease progression of prostate, renal, or bladder tumors present in a sample. The methods involve providing a sample from a human individual, wherein in the sample comprises genetic material from a prostate, renal, or bladder tumor and analyzing the genetic material to determine if there is an alteration in at least one distinct genomic region is selected from the group consisting of the distinct genomic regions set forth at least one of Tables 3-8. The alteration is correlated to the diagnosis or prognosis of prostate, renal, or bladder tumors. In certain embodiments of the methods of the invention, analyzing the genetic material comprises: (i) providing a microarray comprising a substrate with a plurality of nucleic acid molecules corresponding to distinct genomic regions arrayed thereon, wherein each of the nucleic acid molecules corresponding to distinct genomic regions is individually capable of hybridizing to material present in the sample, and wherein the genomic regions arrayed on the substrate are regions wherein an alteration therein is correlated to the diagnosis or prognosis of prostate, renal, or bladder tumors; (ii) labeling genetic material from the sample; and (iii) hybridizing the labeled genetic material with the genomic regions arrayed on the substrate. In other embodiments of the invention, analyzing the genetic material can comprise at least one of the technologies set forth in Table 2.

Non-limiting embodiments of the invention include, for example, the following embodiments.

1. A method of diagnosing renal cortical neoplasms, the method comprising:
(a) analyzing the genetic material from a sample obtained from a human individual to determine the presence or absence of the chromosomal aberrations set forth in FIG. 3 and/or Table 12; and
(b) classifying the subtype of renal cortical neoplasm in the sample using the decision tree shown in FIG. 3.

2. The method of embodiment 1, wherein the presence or absence one or more of the chromosomal aberrations is determined using the criteria set forth in Table 12.

3. The method of embodiment 1 or 2, wherein step (a) comprises:
(i) providing a microarray, said microarray comprising a substrate with a plurality of nucleic acid molecules corresponding to distinct genomic regions arrayed thereon, wherein each of the nucleic acid molecules is individually capable of hybridizing to material present in said sample, and wherein the genomic regions arrayed on the substrate are regions wherein an alteration therein is correlated to the diagnosis or prognosis of prostate, renal, or bladder tumors.
(ii) labeling said genetic material or portion thereof; and
(iii) hybridizing the labeled genetic material or portion thereof with the genomic regions arrayed on the substrate.

4. The method of any one of embodiments 1-3, wherein analyzing comprises targeted array comparative genomic hybridization (array CGH) or whole genome array CGH.

5. The method of embodiment 3 or 4, wherein said analyzing said genetic material comprises analyzing the hybridization pattern of said labeled genetic material to said genomic regions to detect the presence of alterations in said genetic material from said sample.

6. The method of any one of embodiments 3-5, wherein at least one of the distinct genomic regions is selected from the group consisting of: 1p36.32-p36.33, 1p35.2-p36.11, 1p32.3-p33, 1p21.3-p22.2, 1q21.1-q23.1, 1q25.3-q31.2, 1q32.1-q32.3, 1q41, 1q42.2, 2p25.1, 2p23.3-p24.1, 2p22.1-p22.2, 2q14.2-q14.3, 2q22.1-q22.3, 2q34-q35, 2q37.3, 3p25.1-p26.1, 3p21.2-p21.31, 3p13-p14.2, 3q13.33-q21.2, 3q26.2-q26.31, 3q26.32-q26.33, 3q28-q29, 4p16.2-p16.3, 4p12-p14, 4q28.1-q28.3, 4q34.1-q35.2, 5p15.33, 5p15.31-p15.1, 5p13.3-p13.1, 5q11.2, 5q14.1-q14.3, 5q21.2-q22.1 5q23.1-q23.2, 5q35.1-q35.3, 6p23-p25.2, 6p22.3, 6p21.31-p22.1, 6q14.2-q16.1, 6q16.3q-21, 6q24.3-q25.3, 7p22.3-p21.3, 7p21.2-p21.3, 7p14.3-p15.2, 7p11.2-p12.1, 7q11.22-q21.11, 7q31.31-q31.2, 7q36.1-q36.2, 8p23.2-p23.3, 8p21.2-p22, 8p11.21-p12, 8q13.3-q21.13, 8q22.1-q22.3, 8q24.13-q24.21, 9p24.2-p24.1, 9p21.2-p21.3, 9p13.2-p21.1, 9q21.31-q21.33, 9q33.2-q33.3, 10p13-p15.3, 10q23.2-q23.31, 10q25.1-q25.3, 11p15.3-p15.4, 11p12, 11q13, 11q14.1-q14.2, 11q22.3-q23.3, 12p12.3-p13.31, 12q13.2-q14.1, 12q24.21-q24.31, 13q11-q12.2, 13q13.3, 13q14.11-q14.3, 13q21.2-q22.1, 13q32.3-q34, 14q11.2, 14q23.1-q23.3, 14q32.13-q32.33, 15q23-q24.1, 15q25.2-q26.1, 16p13.12-p13.3, 16p12.2-p12.3, 16q21-q23.3, 17p13.1-p13.3, 17p11.2-p12, 17q12-q21.31, 17q25.1-q25.3, 18p11.31-p11.32, 18q11.2, 18q21.1-q23, 19p13.3, 19q13.32-q13.41, 20p12.1-p12.3, 20q13.2-q13.33, 21q22.13-q22.3, 22q11.1-g12.1, 22q13.2-q13.32, Xp11.22-p11.23, Xq13.2-q13.3, Xq21.33-q22.1, and Yp11.1-p11.2.

7. The method of embodiment 6, wherein the plurality of nucleic acid molecules comprises nucleic acid molecules corresponding to at least 10, 11, 12, 13, 14, 15, 16, 17, 18, 19, 20, 25, 30, or all of the distinct genomic regions set forth in embodiment 6.

8. The method of embodiment 6, wherein the plurality of nucleic acid molecules essentially consists of or consists of nucleic acid molecules corresponding to 10, 11, 12, 13, 14, 15, 16, 17, 18, 19, 20, 25, 30 or all of the distinct genomic regions set forth in embodiment 6.

9. The method of any one of embodiments 3-8, wherein the plurality of nucleic acid molecules comprises, essentially consists of, or consists of the nucleic acid molecules corresponding to the distinct genomic regions set forth in Table 3.

10. The method of any one of embodiments 1-9, wherein the subtypes are selected from the group consisting of clear cell renal cell carcinoma (ccRCC), papillary renal cell carcinoma (pRCC), and chromophobe renal cell carcinoma (chrRCC), oncocytoma (OC), Not-classifiable neoplasm, and Benign.

11. The method of any one of embodiments 1-10, wherein the subtypes are clear cell renal cell carcinoma (ccRCC), papillary renal cell carcinoma (pRCC), and chromophobe renal cell carcinoma (chrRCC), oncocytoma (OC), Not-classifiable neoplasm, and Benign.

12. The method of any one of embodiments 1-11, wherein the aberrations comprise, essentially consist of, or consist of the loss of VHL gene, the loss of chr2, the gain of 17q, the gain of chr7, the gain of chr12, the gain of 16p, the gain of 20q, the gain of 5qter, the gain of chr3, the loss of chr6, the loss of chr10, the loss of chr17, the loss of 8p, the partial or entire loss of chr1, and the loss of 3p21.2-21.31.

13. The method of any one of embodiments 1-12, wherein analyzing comprises array CGH, wherein the method further comprises analyzing the genetic material to determine then presence or absence of rearrangements at the CCND1 (11q13) locus, and wherein the presence of the CCND1 rearrangements is indicative of OC.

14. The method of any one of embodiments 1, 2, and 10-13, wherein analyzing comprises next-generation sequencing.

15. The method of any one of embodiments 1-14, wherein the method further comprises analyzing the genetic material to determine then presence or absence of rearrangements at the CCND1 (11q13) locus, and wherein the presence of the CCND1 rearrangements is indicative of OC.

16. The method of any one of embodiments 1-15, wherein the sample is from a human individual previously diagnosed as having a small renal mass.

17. The method any one of embodiments 1-16, wherein the sample comprises the small renal mass.

18. The method of any one of embodiments 1-17, wherein the sample is selected from the group consisting of a frozen resected sample, a needle biopsy sample, and a fresh resected sample.

19. The method of any one of embodiments 1-18, wherein a computer is used in analyzing the genetic material in step (a) or in classifying the subtype in step (b), or in both step (a) and step (b).

20. The method of any one of embodiments 1-19, wherein the decision tree is embodied in computer software.

21. A kit comprising a microarray suitable for the detection of genomic aberrations and the decision tree set forth in FIG. 3, wherein the aberrations comprise, essentially consist of, or consist of the loss of VHL gene, the loss of chr2, the gain of 17q, the gain of chr7, the gain of chr12, the gain of 16p, the gain of 20q, the gain of 5qter, the gain of chr3, the loss of chr6, the loss of chr10, the loss of chr17, the loss of 8p, the partial or entire loss of chr1, and the loss of 3p21.2-21.31.

22. The kit of embodiment 21, wherein the decision tree is on printed material or in a computer-readable form.

23. The kit of embodiment 21 or 22, wherein the decision tree is embodied in computer software.

24. The kit of any one of embodiments 21-23, further comprising the criteria set forth in Table 12 for determining the presence or absence of one or more of the chromosomal aberrations, wherein the criteria are set forth in printed material, in a computer-readable form, or embodied in computer software.

25. The kit of embodiment 24, further comprising instructions for using the kit to detect the genomic aberrations, wherein the instructions are set forth in printed material, in computer-readable form, or embodied in computer software.

26. The kit any one of embodiments 21-25, further comprising instructions for classifying the subtype of renal cortical neoplasm in a sample comprising genetic material from a human individual.

27. The kit of embodiment 26, wherein the subtypes comprise, essentially consist of, or consist of clear cell renal cell carcinoma (ccRCC), papillary renal cell carcinoma (pRCC), and chromophobe renal cell carcinoma (chrRCC), oncocytoma (OC), Not-classifiable neoplasm, and Benign.

28. The kit of any one of embodiments 21-27, wherein the microarray comprises a plurality of nucleic acid molecules corresponding to distinct genomic regions and at least one of the distinct genomic regions is selected from the group consisting of: 1p36.32-p36.33, 1p35.2-p36.11, 1p32.3-p33, 1p21.3-p22.2, 1q21.1-q23.1, 1q25.3-q31.2, 1q32.1-q32.3, 1q41, 1q42.2, 2p25.1, 2p23.3-p24.1, 2p22.1-p22.2, 2q14.2-q14.3, 2q22.1-q22.3, 2q34-q35, 2q37.3, 3p25.1-p26.1, 3p21.2-p21.31, 3p13-p14.2, 3q13.33-q21.2, 3q26.2-q26.31, 3q26.32-q26.33, 3q28-q29, 4p16.2-p16.3, 4p12-p14, 4q28.1-q28.3, 4q34.1-q35.2, 5p15.33, 5p15.31-p15.1, 5p13.3-p13.1, 5q11.2, 5q14.1-q14.3, 5q21.2-q22.1 5q23.1-q23.2, 5q35.1-q35.3, 6p23-p25.2, 6p22.3, 6p21.31-p22.1, 6q14.2-q16.1, 6q16.3q-21, 6q24.3-q25.3, 7p22.3-p21.3, 7p21.2-p21.3, 7p14.3-p15.2, 7p11.2-p12.1, 7q11.22-q21.11, 7q31.31-q31.2, 7q36.1-q36.2, 8p23.2-p23.3, 8p21.2-p22, 8p11.21-p12, 8q13.3-q21.13, 8q22.1-q22.3, 8q24.13-q24.21, 9p24.2-p24.1, 9p21.2-p21.3, 9p13.2-p21.1, 9q21.31-q21.33, 9q33.2-q33.3, 10p13-p15.3, 10q23.2-q23.31, 10q25.1-q25.3, 11p15.3-p15.4, 11p12, 11q13, 11q14.1-q14.2, 11q22.3-q23.3, 12p12.3-p13.31, 12q13.2-q14.1, 12q24.21-q24.31, 13q11-q12.2, 13q13.3, 13q14.11-q14.3, 13q21.2-q22.1, 13q32.3-q34, 14q11.2, 14q23.1-q23.3, 14q32.13-q32.33, 15q23-q24.1, 15q25.2-q26.1, 16p13.12-p13.3, 16p12.2-p12.3, 16q21-q23.3, 17p13.1-p13.3, 17p11.2-p12, 17q12-q21.31, 17q25.1-q25.3, 18p11.31-p11.32, 18q11.2, 18q21.1-q23, 19p13.3, 19q13.32-q13.41, 20p12.1-p12.3, 20q13.2-q13.33, 21q22.13-q22.3, 22 q11.1-q12.1, 22q13.2-q13.32, Xp11.22-p11.23, Xq13.2-q13.3, Xq21.33-q22.1, and Yp11.1-p11.2.

29. The kit of embodiment 28, wherein the plurality of nucleic acid molecules comprises nucleic acid molecules corresponding to at least 10, 11, 12, 13, 14, 15, 16, 17, 18, 19, 20, 25, 30 or all of the distinct genomic regions set forth in embodiment 26.

30. The kit of embodiment 28, wherein the plurality of nucleic acid molecules essentially consists of or consists of nucleic acid molecules corresponding to 10, 11, 12, 13, 14, 15, 16, 17, 18, 19, 20, 25, 30, or all of the distinct genomic regions set forth in embodiment 26.

31. The kit of any one of embodiments 21-30, wherein the microarray comprises a plurality of nucleic acid molecules corresponding to distinct genomic regions, the plurality of nucleic acid molecules comprising, essentially consisting of, or consisting of nucleic acid molecules corresponding to the distinct genomic regions set forth in Table 3.

32. A microarray for determining the type or predicting the disease progression of prostate, renal, or bladder tumors present in a sample, said microarray comprising a substrate with a plurality of nucleic acid molecules corresponding to distinct genomic regions arrayed thereon, wherein an alteration in at least one of said distinct genomic regions is correlated to the diagnosis or prognosis of one or more types of tumors mentioned above, and wherein said at least one distinct genomic region is selected from the group consisting of the distinct genomic regions set forth in at least one of Tables 3-8.

33. The microarray of embodiment 32, wherein at least one of the distinct genomic regions is selected from the group consisting of: 1p36.32-p36.33, 1p35.2-p36.11, 1p32.3-p33, 1p21.3-p22.2, 1q21.1-q23.1, 1q25.3-q31.2, 1q32.1-q32.3, 1q41, 1q42.2, 2p25.1, 2p23.3-p24.1, 2p22.1-p22.2, 2q14.2-q14.3, 2q22.1-q22.3, 2q34-q35, 2q37.3, 3p25.1-p26.1, 3p21.2-p21.31, 3p13-p14.2, 3q13.33-q21.2, 3q26.2-q26.31, 3q26.32-q26.33, 3q28-q29, 4p16.2-p16.3, 4p12-p14, 4q28.1-q28.3, 4q34.1-q35.2, 5p15.33, 5p15.31-p15.1, 5p13.3-p13.1, 5q11.2, 5q14.1-q14.3, 5q21.2-q22.1 5q23.1-q23.2, 5q35.1-q35.3, 6p23-p25.2, 6p22.3, 6p21.31-p22.1, 6q14.2-q16.1, 6q16.3q-21, 6q24.3-q25.3, 7p22.3-p21.3, 7p21.2-p21.3, 7p14.3-p15.2, 7p11.2-p12.1, 7q11.22-g21.11, 7q31.31-q31.2, 7q36.1-q36.2, 8p23.2-p23.3, 8p21.2-p22, 8p11.21-p12, 8q13.3-q21.13, 8q22.1-q22.3, 8q24.13-q24.21, 9p24.2-p24.1, 9p21.2-p21.3, 9p13.2-p21.1, 9q21.31-q21.33, 9q33.2-q33.3, 10p13-p15.3, 10q23.2-q23.31, 10q25.1-q25.3, 11p15.3-p15.4, 11p12, 11q13, 11q14.1-q14.2, 11q22.3-q23.3, 12p12.3-p13.31, 12q13.2-q14.1, 12q24.21-q24.31, 13q11-q12.2, 13q13.3, 13q14.11-q14.3, 13q21.2-q22.1, 13q32.3-q34, 14q11.2, 14q23.1-q23.3, 14q32.13-g32.33, 15q23-q24.1, 15q25.2-q26.1, 16p13.12-p13.3, 16p12.2-p12.3, 16q21-q23.3, 17p13.1-p13.3, 17p11.2-p12, 17q12-q21.31, 17q25.1-q25.3, 18p11.31-p11.32, 18q11.2, 18q21.1-q23, 19p13.3, 19q13.32-q13.41, 20p12.1-p12.3, 20q13.2-q13.33, 21q22.13-q22.3, 22q11.1-q12.1, 22q13.2-q13.32, Xp11.22-p11.23, Xq13.2-q13.3, Xq21.33-q22.1, and Yp11.1-p11.2.

34. The microarray of embodiment 33, wherein the plurality of nucleic acid molecules comprises, essentially consists of, or consists of nucleic acid molecules corresponding to at least 2, 3, 4, 5, 6, 7, 8, 9, 10, 11, 12, 13, 14, 15, 16, 17, 18, 19, 20, 25, 30, or all of the distinct genomic regions set forth in embodiment 32.

35. The microarray of any one of embodiments 31-34, wherein the plurality of nucleic acid molecules comprises, essentially consists of, or consists of the nucleic acid molecules corresponding to the distinct genomic regions set forth in Table 3.

### BRIEF DESCRIPTION OF THE FIGURES

FIG. 1. A preliminary decision tree for the classification of renal cortical neoplasm subtypes analyzed by array CGH. The subtypes shown in the preliminary decision tree that are classified as a renal cell carcinoma (RCC) are clear cell renal cell carcinoma (ccRCC), papillary renal cell carcinoma (pRCC), and chromophobe renal cell carcinoma (chrRCC). Also shown in the decision tree are the benign renal cortical neoplasm subtype, oncocytoma (OC), Non-RCC neoplasm (i.e. not classifiable), and Normal (i.e. benign).

FIG. 2. TCGA data analysis to identify diagnostic markers for classification of malignant RCC subtypes. Sequential stratification of predominant genomic markers found in the TCGA RCC copy number dataset (n=629) and preliminary subtyping based on the presence or absence of four essential CNAs: loss of VHL gene, gain of 5qter, loss of chr17 and gain of 17q.

FIG. 3. Decision tree algorithm for copy number-based subtyping of renal tumors. Schematic representation of step-wise classification of ccRCC, pRCC, chrRCC and OC based on 15 diagnostic CNAs identified by TCGA dataset and published literature. The underlined criteria in final pRCC decision point indicate the change made to original tree in order to increase the efficiency of OC subtyping. If the no alteration was detected, the specimen was regarded as benign. If alterations other than the 15 CNAs were observed, the specimen was stratified as not-classifiable.

FIG. 4. CCND1 break-apart FISH. Representative FISH image of nuclei from an OC specimen (CGI-021) showing 11q13 rearrangement with separated 5′-CCND1 (red) and 3′-CCND1 (green) signals and one intact normal fusion (yellow [F]) signal.

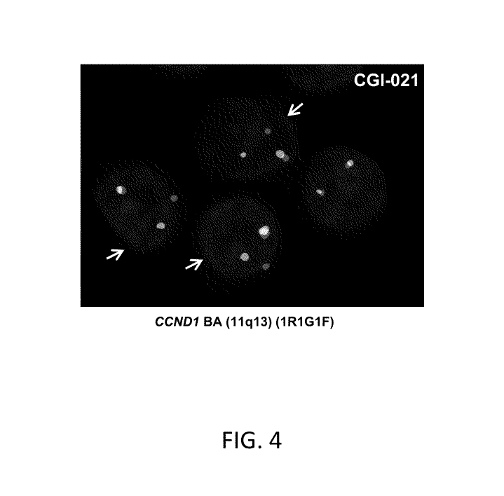

### DETAILED DESCRIPTION OF THE INVENTION

The present invention now will be described more fully hereinafter with reference to accompanying drawings or figures, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

The article “a” and “an” are used herein to refer to one or more than one (i.e., to at least one) of the grammatical object of the article. By way of example, “an element” means one or more element.

Throughout the specification the word “comprising,” or variations such as “comprise” or “comprises,” will be understood to imply the inclusion of a stated element, integer or step, or group of elements, integers or steps, but not the exclusion of any other element, integer or step, or group of elements, integers or steps.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Various technical and scientific terms are used in the present disclosure, and the meaning of said terms is understood to be as expressly defined herein or as otherwise ascertainable from the context of the present disclosure. To the extend such terms are not expressly or inherently defined herein, the meaning of such terms is understood to be the same as commonly understood by one of ordinary skill in the art to which this invention belongs.

As used herein, the term “genomic region” is intended to mean a portion of nucleic acid polymer that is contained within the human genome complement. The term can relate to a specific length of DNA. The term can also be used in relation to specific oligonucleotides corresponding to a genomic region or part thereof. Location of the nucleic acid polymer within the genome can be defined with respect to either the chromosomal band in the human genome or one or more specific nucleotide positions in the human genome.

As used herein, the terms “genitourinary cancer”, “genitourinary malignancy”, or urogenital cancer” is intended to mean a urogenital malignancy arising in the prostate, kidney, or bladder.

As used herein, the term “genomic aberration” is intended to mean any aberration, alteration or change in the genome of an individual from the wild-type genome, particularly a change that is associated or correlated with a particular cancer or cancerous subtype or benign neoplasm. Such aberrations, alteration or changes can include, for example, gains or losses of entire chromosomes or arms or parts thereof, losses or gains in all or parts of genes or in regions of the genome not known to comprise genes. More preferably, such aberrations, alteration or changes can include gains or losses of entire chromosomes or arms or parts thereof, losses or gains in all or parts of genes or in regions of the genome not known to comprise genes. used herein, the term “prostate cancer” is intended to mean an abnormal growth of the tissue cells within the human male prostate gland.

As used herein, the term “kidney cancer” or “renal cancer” is intended to refer to all cancer within the human kidney that includes cancers originating from the renal epithelium within the kidney and other parts of the kidney. This includes renal cortical neoplasms comprising malignant and benign subtypes. Malignant subtypes include renal cell carcinomas (RCC), specifically clear cell RCC, papillary RCC, and chromophobe RCC. Benign subtype includes renal oncocytoma.

As used herein, the terms “bladder tumor” is intended to refer to cancer originating from the bladder which can be either invasive or non-invasive.

As used herein, the terms “tumor” and “neoplasm” are equivalent terms that are used interchangeably herein and refer to abnormal growth of body tissue. Tumors or neoplasms can be cancerous (i.e., malignant) or noncancerous (i.e., benign).

As used herein, the terms “biopsy” and “biopsy specimen” are intended to mean a biological sample of tissue, cells, or liquid taken from the human body.

As used herein, the term “sample” is intended to mean a biological sample of tissue, cells, or liquid taken from the body of a human individual and comprises genetic material of the individual unless expressly stated otherwise or apparent from the context of usage. Preferrably, a sample of the present invention comprises genomic DNA of the individual. More preferably, a sample of the present invention comprises the entire nuclear genome present in tissue and/or cells from which the sample is derived. It is recognized that a sample from human tumor cells or tissues might not possess the entire human nuclear genome when compared to a sample from human cells or tissues that are non-cancerous because losses of whole or parts of chromosomes might have occurred.

As used herein, the term “genetic material” is intended to mean materials comprising or formed predominately of nucleic acids. The term specifically is intended to encompass, deoxyribonucleic acids (DNA) or fragments thereof and ribonucleic acids (RNA) or fragments thereof. The term also may be used in reference to genes, chromosomes, and/or oligonucleotides and may encompass any portion of the nuclear genome and/or the mitochondrial genome of the human body.

As used herein, the term “label” is intended to mean any substance that can be attached to genetic material so that when the genetic material binds to a corresponding site a signal is emitted or the labeled probe can be detected by a human observer or an analytical instrument. Labels envisioned by the present invention can include any labels that emit a signal and allow for identification of a component in a sample or reference genetic material. Non-limiting examples of labels encompassed by the present invention include fluorescent moieties, radioactive moieties, chromogenic moieties, and enzymatic moieties.

As used herein, the term “probe” is intended to mean any molecular structure or substructure that hybridizes or otherwise binds to a genomic region.

As used herein, a “genome scanning technology” is an array-based technology that can be used for the karyotyping of inherited and somatic chromosomal aberrations. Genome scanning technologies include, but are not limited to, single nucleotide polymorphism arrays (SNP-As), copy number oligonucleotide arrays (oligo-As), and comparative genome hybridization arrays (array CGH) with oligonucleotide and bacterial artificial chromosome (BAC) probes.

Chromosome abnormalities are found in cancer, and include genomic rearrangement, gain/amplification, deletion (loss), uniparental disomy, and mutation. These alterations can affect gene expression (and hence function) affecting multiple disease types. The detection and molecular definition of these alterations has stimulated research directed at understanding not only the functional role of the involved gene(s) in disease etiology but also in normal human biology. Preferred alterations for the present invention are gains, losses, and rearrangements.

The present invention provides a tool that can be used in the diagnosis and prognosis of cancers originated from prostate, kidney, and bladder. The tool is particularly beneficial because it can be used in new methodologies that utilize minimal available biopsy material, can be carried out with an analyte that is stable over time, and is less invasive than known procedures for diagnostic/prognostic purposes.

In certain embodiments, the tool of the present invention can be a microarray for detecting (and thus diagnosing and stratifying) particular genomic aberrations within prostate tumor, renal tumor within kidney or bladder tumor present in a sample. Particularly, the microarray may employ comparative genomic hybridization (array-CGH) to assist in the diagnosis and prognosis of prostate, renal, and bladder tumors. Array-CGH is a useful diagnostic tool because it can utilize DNA from fresh, frozen, or formalin-fixed paraffin-embedded (FFPE) specimens and can, in array format, detect genomic gain/loss at a large number of chromosomal loci at one time. The present invention provides an exemplary instance for use of such methods in the areas of prostate, renal, or bladder cancer diagnosis and prognosis in a clinical setting.

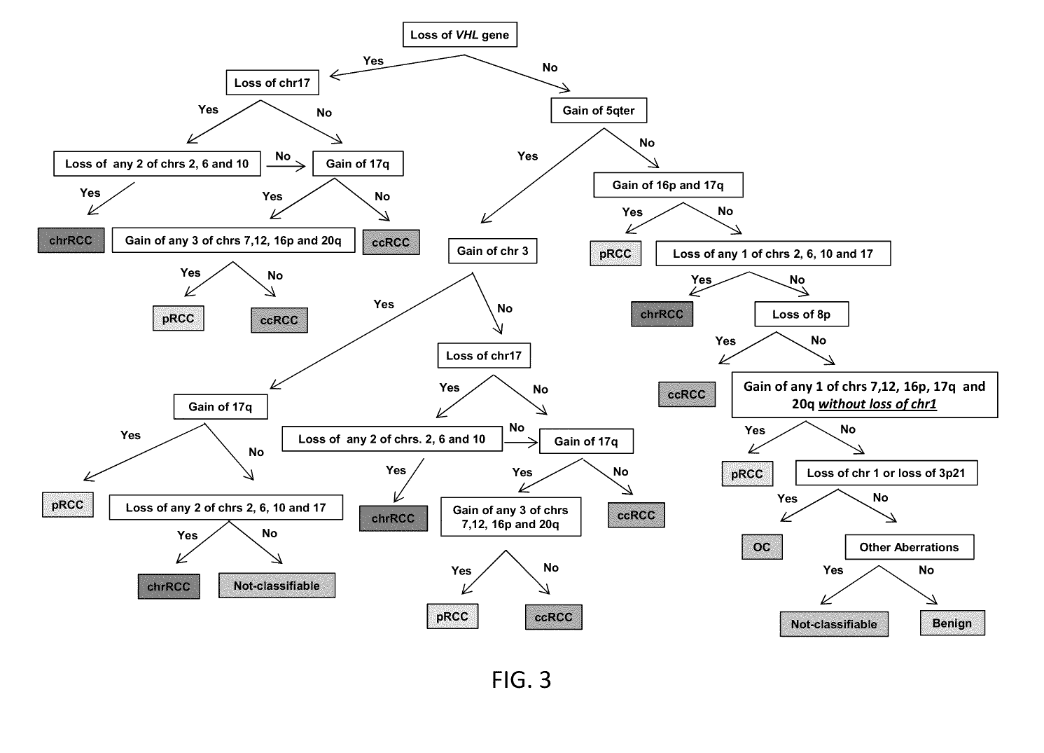

In particular embodiments, the present invention provides a specific oligonucleotide-based urogenital cancer array (UroGenRA®) that is useful in diagnosis and prognosis of prostate, renal, and bladder cancers. The urogenital cancer array can represent a plurality of distinct genomic regions that exhibit an alteration therein (e.g., gain and/or loss) in the prostate, renal, or bladder tumors and can be used in varying techniques, platforms, and statistical algorithms. In specific embodiments, the invention provides technical criteria for alteration detection in available biopsy material. In other embodiments, the invention provides methods wherein prostate, renal or bladder tumors can be submitted to UroGenRA® array CGH and alterations correlated to specific disease indicators and/or outcomes of the prostate, renal, or bladder tumors. Further, the invention provides a decision tree (FIG. 3) for diagnosis of subtypes of renal cortical neoplasms. Accordingly, the diagnostic tools of the present invention, such as UroGenRA®, are useful in prostate, renal, and bladder diagnosis/prognosis and can be easily integrated into current treatment regimens.

In one aspect, the present invention specifically provides a microarray for diagnosing the type of urogenital tumor present in a sample. The microarray may be an oligonucleotide array and can be characterized by the inclusion of genomic regions wherein an alteration in the genomic region is consistent with one or more specific types of renal tumors. More particularly, the genomic regions represented on the microarray may be regions wherein a copy number aberration (CNA) (e.g., gain, loss, or both gain and loss) in the region is consistent with one or more specific types of renal cell carcinomas. In other words, the genomic regions included in the microarray of the present invention may be regions wherein genomic CNAs are shown to be common to specific types of renal tumors. As more fully described herein, the microarray thus is useful in the diagnosis/prognosis as well as classification of different tumor subtypes.

In one embodiment, a microarray according to the invention may comprise a substrate with a plurality of nucleic acid molecules corresponding to distinct genomic regions arrayed thereon. Any substrate useful in forming diagnostic arrays may be used according to the present invention. For example, glass substrates, such as glass slides, may be used. Other non-limiting examples of useful substrates include silicon-based substrates, metal incorporating substrates (e.g., gold and metal oxides, such as titanium dioxide), gels, and polymeric materials. Useful substrates may be functionalized, such as to provide a specific charge, charge density, or functional group present at the substrate surface for immobilization of materials (e.g., oligonucleotides) to the substrate.

Preferably, each of the nucleic acid molecules corresponding to distinct genomic regions represented on the microarray is individually capable of hybridizing to material present in a sample (test and/or reference). In certain embodiments, the test sample may comprise all or part of a biopsy or biopsy specimen. In other embodiments, the test sample may comprise tissue that is fresh, frozen, or formalin-fixed paraffin-embedded (FFPE). In further embodiments, the test sample may comprise all or part of a biopsy specimen, including tissue, core biopsy, or fine needle aspirate. The test sample particularly may comprise genetic material. Preferably, the test sample comprises material in some form capable of hybridizing to the genomic regions represented on the microarray of the present invention. In specific embodiments, the test sample may comprise DNA or fragments thereof.

The methods of the present invention involve the use of genetic material of a sample from a human individual. It is recognized that the methods of the present invention do not depend on using all of the genetic material in the sample. Accordingly, any reference herein to “genetic material” or “the genetic material” from a sample is not intended to a mean all of the genetic material in the sample unless expressly stated or otherwise apparent from the context of usage. Typically, for the methods disclosed herein, the genetic material comprises at least a portion of the genomic DNA in the sample. Preferably, such a portion of the genomic DNA is representative of the all of the genomic DNA in the sample.

In specific embodiments, the genomic regions arrayed on the substrate can be regions wherein a particular alteration therein is correlated to one or more disease indicators or subtypes of prostate, renal, or bladder tumors. The type of alteration identified can be any alteration, as otherwise described herein, that is correlated to a specific type of tumors. In specific embodiments, the alteration can be a copy number aberration, particularly a gain or a loss of at least a portion of a distinct genomic region.

The microarrays of the present invention provide a plurality of genomic regions, and the exact number of genomic regions can vary depending upon the desired use of the microarray (e.g., diagnostic versus prognostic), the desired specificity of the array, and other desired outcomes. Preferably, the microarray comprises a sufficient number of genomic regions to identify a specific disease indicator of prostate, renal, or bladder tumors that may be represented within the test sample. In particular embodiments, a microarray according to the present invention includes a number of genomic regions sufficient to identify the presence in a sample of one or more disease indicators of prostate, renal or bladder tumors. The microarray of the invention may comprise only a single genomic region useful to identify a single type of tumor (diagnosis) or outcome (prognosis). Preferably, the microarrays of the present invention comprise a plurality of genomic regions that each can be useful to identify a single type of tumor or outcome. As some genomic regions that may be used according to the invention can correlate to two or more different types of tumors, it can be useful according to the invention for the microarray to include many different genomic regions having different alterations that correlate to various aspects of prostate, renal, or bladder tumors to assist in interpretation of signaling to identify the specific type or types of prostate, renal or bladder tumors that are identified via the test sample.

The exact number of different genomic regions represented on the microarrays of the present invention can vary based upon the desired outcome of the test in which the array may be used. In specific embodiments, a single microarray according to the invention may comprise at least 2 different genomic regions, at least 5 different genomic regions, at least 10 different genomic regions, at least 15 different genomic regions, at least 20 different genomic regions, at least 25 different genomic regions, at least 30 different genomic regions, at least 35 different genomic regions, at least 40 different genomic regions, at least 45 different genomic regions, at least 50 different genomic regions, at least 55 different genomic regions, at least 60 different genomic regions, at least 65 different genomic regions, at least 70 different genomic regions, at least 75 different genomic regions, at least 80 different genomic regions, at least 85 different genomic regions, at least 90 different genomic regions, at least 95 different genomic regions or at least 100 different genomic regions. A microarray designed to diagnose only one or two different cancers among prostate, renal, or bladder tumors may use a smaller number of different genomic regions, while a microarray designed to detect many different types of the tumors could include a much larger number of different genomic regions. Further, each different genomic region can be included in the array in multiple copies. The total number of genomic regions provided on a single microarray according to the invention thus can be greater than about 100, greater than about 250, greater than about 500, greater than about 1,000, greater than about 2,500, greater than about 5,000, greater than about 10,000, greater than about 15,000, greater than about 20,000, greater than about 25,000, greater than about 30,000, greater than about 35,000, greater than about 40,000, greater than about 45,000, or greater than about 50,000. In certain embodiments, the total number of genomic regions provided on a single microarray can comprise, or consist of, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100 or more of the different genomic regions set forth in Table 3 below.

In certain other embodiments, the genomic regions used on the microarrays of the present invention may be identified in relation to chromosomal bands (although the region represented on the array need not necessarily include the entire band). Particularly, the plurality of genomic regions can comprise at least one chromosomal band selected from the group shown in Table 3 below. In addition to a varying degree based upon the different regions that may be represented on the microarray, the device of the present invention also may vary based upon probe density within specific regions and multiplicity of arrayed oligonucleotides.

As evident from above, the microarrays of the present invention can be designed to incorporate genomic regions wherein a specific alteration, such as a gain or loss, correlates genetic material hybridized (e.g., DNA or fragments thereof) there with a specific disease feature among prostate, renal or bladder tumors and the overall diagnosis/prognosis of the respective patient. Because of the identification of a large number of different genomic regions that correlate to multiple disease indicators of prostate, renal, or bladder tumors, it is also possible according to the invention to provide a single array (e.g., a single chip or a single slide) to which a test sample can be applied and identify more than one of the specific disease indicator of prostate, renal, or bladder tumors represented in the array and the diagnosis/prognosis of the patient from which the biopsy was derived. Thus, the microarrays of the present invention can provide a clear diagnostic and prognostic purpose.

In addition to the genomic regions described above that are present on the substrate, the microarrays of the present invention further comprise one or more probes that may be useful for normalization of test results or to use as a comparative for analytical purposes. In one embodiment, a “backbone” probe set may be present that covers the entire chromosomal complement. Such backbone probe set may comprise varying numbers of probes at varying levels of resolution and preferably excludes regions of known copy number variation.

TABLE 3
Genomic Regions Represented on the UroGenRA ®
Urogenital Cancer Array

| Region | Location (Mbp)* | Size (Mbp) |
|---|---|---|
| 1p36.32-36.33 | chr1: 0-4 | 4 |
| 1p35.2-36.11 | chr1: 26-31 | 5 |
| 1p32.3-33 | chr1: 48-55 | 7 |
| 1p21.3-22.2 | chr1: 92-95 | 3 |
| 1q21.1-23.1 | chr1: 145-158 | 13 |
| 1q25.3-31.2 | chr1: 181-192 | 11 |
| 1q32.1-32.3 | chr1: 207-212 | 5 |
| 1q41 | chr1: 217-221 | 4 |
| 1q42.2 | chr1: 232-234 | 2 |
| 2p25.1 | chr2: 9-12 | 3 |
| 2p23.3-24.1 | chr2: 22-27 | 5 |
| 2p22.1-22.2 | chr2: 37-39 | 2 |
| 2q14.2-14.3 | chr2: 121-128 | 7 |
| 2q22.1-22.3 | chr2: 138-145 | 7 |
| 2q34-35 | chr2: 214-221 | 7 |
| 2q37.3 | chr2: 238-243 | 5 |
| 3p25.1-26.1 | chr3: 8-15 | 7 |
| 3p21.2-21.31 | chr3: 45-51 | 6 |
| 3p13-14.2 | chr3: 60-72 | 12 |
| 3q13.33-21.2 | chr3: 120-124 | 4 |
| 3q26.2-26.31 | chr3: 168-172 | 4 |
| 3q26.32-26.33 | chr3: 176-182 | 6 |
| 3q28-29 | chr3: 190-196 | 6 |
| 4p16.2-16.3 | chr4: 0-5 | 5 |
| 4p12-14 | chr4: 41-46 | 5 |
| 4q28.1-28.3 | chr4: 124-137 | 13 |
| 4q34.1-35.2 | chr4: 172-188 | 16 |
| 5p15.33 | chr5: 0-4.5 | 5 |
| 5p15.31-5p15.1 | chr5: 7.7-16 | 8.3 |
| 5p13.3-p13.1 | chr5: 32-41 | 9 |
| 5q11.2 | chr5: 55-60 | 5 |
| 5q14.1-14.3 | chr5: 82-85 | 3 |
| 5q21.2-22.1 | chr5: 96-109 | 13 |
| 5q23.1-23.2 | chr5: 116-122 | 6 |
| 5q35.1-35.3 | chr5: 171-181 | 10 |
| 6p23-25.2 | chr6: 4-13.5 | 10 |
| 6p22.3 | chr6: 17-23 | 6 |
| 6p21.31-22.1 | chr6: 30-36 | 6 |
| 6q14.2-16.1 | chr6: 84-94 | 10 |
| 6q16.3-21 | chr6: 104-114 | 10 |
| 6q24.3-25.3 | chr6: 149-161 | 12 |
| 7p22.3-21.3 | chr7: 2.5-7.5 | 5 |
| 7p21.2-21.3 | chr7: 10-14 | 4 |
| 7p14.3-15.2 | chr7: 28-32 | 7 |
| 7p11.2-12.1 | chr7: 53-57 | 4 |
| 7q11.22-21.11 | chr7: 66-81 | 15 |
| 7q31.31-31.2 | chr7: 115-119 | 4 |
| 7q36.1-36.2 | chr7: 148-154 | 6 |
| 8p23.2-23.3 | chr8: 1-5 | 4 |
| 8p21.2-22 | chr8: 19-27 | 8 |
| 8p11.21-12 | chr8: 32-40 | 8 |
| 8q13.3-21.13 | chr8: 70-82 | 12 |
| 8q22.1-22.3 | chr8: 99-103 | 4 |
| 8q24.13-24.21 | chr8: 124-131 | 7 |
| 9p24.2-p24.1 | chr9: 4-6 | 2 |
| 9p21.2-p21.3 | chr9: 21-27 | 6 |
| 9p13.2-21.1 | chr9: 33-37 | 4 |
| 9q21.31-21.33 | chr9: 84-88 | 4 |
| 9q33.2-q33.3 | chr9: 124-130 | 6 |
| 10p13-15.3 | chr10: 0-17 | 17 |
| 10q23.2-23.31 | chr10: 88-92 | 4 |
| 10q25.1-25.3 | chr10: 106-119 | 13 |
| 11p15.3-15.4 | chr11: 3-11 | 8 |
| 11p12 | chr11: 35-40 | 5 |
| 11q13 | chr11: 66-76 | 10 |
| 11q14.1-14.2 | chr11: 82-86 | 4 |
| 11q22.3-23.3 | chr11: 110-120 | 10 |
| 12p12.3-13.31 | chr12: 10-15 | 5 |
| 12q13.2-14.1 | chr12: 55-61 | 6 |
| 12q24.21-24.31 | chr12: 116-123 | 7 |
| 13q11-12.2 | chr13: 18-23 | 5 |
| 13q13.3 | chr13: 36-39 | 3 |
| 13q14.11-14.3 | chr13: 44-52 | 8 |
| 13q21.2-22.1 | chr13: 61-74 | 13 |
| 13q32.3-34 | chr13: 109-114 | 5 |
| 14q11.2 | chr14: 19-21 | 2 |
| 14q23.1-23.3 | chr14: 59-67 | 8 |
| 14q32.13-32.33 | chr14: 95-105 | 10 |
| 15q23-24.1 | chr15: 71-75 | 4 |
| 15q25.2-26.1 | chr15: 85-92 | 7 |
| 16p13.12-13.3 | chr16: 1-13 | 12 |
| 16p12.2-12.3 | chr16: 21-24 | 3 |
| 16q21-23.3 | chr16: 65-82 | 17 |
| 17p13.1-13.3 | chr17: 0-9 | 9 |
| 17p11.2-12 | chr17: 14-17 | 3 |
| 17q12-21.31 | chr17: 38-42 | 4 |
| 17q25.1-25.3 | chr17: 74-78 | 4 |
| 18p11.31-11.32 | chr18: 0-5 | 5 |
| 18q11.2 | chr18: 21-23 | 2 |
| 18q21.1-23 | chr18: 48-76 | 28 |
| 19p13.3 | chr19: 1-5 | 4 |
| 19q13.32-13.41 | chr19: 46-53 | 7 |
| 20p12.1-12.3 | chr20: 9-13 | 5 |
| 20q13.2-13.33 | chr20: 55-62 | 8 |
| 21q22.13-22.3 | chr21: 38-47 | 9 |
| 22q11.1-12.1 | chr22: 16-26 | 10 |
| 22q13.2-13.32 | chr22: 42-49 | 7 |
| Xp11.22-11.23 | chrX: 48-53 | 5 |
| Xq13.2-13.3 | chrX: 72-76 | 4 |
| Xq21.33-22.1 | chrX: 94-101 | 7 |
| Yp11.1-11.2 | chrY: 6-12 | 6 |

*The region represented on the array does not necessarily include the entire band. The band information is obtained from UCSC Genome Assembly February 2009 (GRCh37/hg19).

In a further aspect, the present invention provides methods for diagnosis and prognosis of prostate, kidney, and bladder cancers present in a sample. Tables 4-7 show correlations between CNAs at specific genomic regions and the three types of urogenital malignancies including outcome. Table 4 lists the regions represented in the urogenital cancer array which are differentially altered between the three malignant and one benign renal cortical neoplasms and can be used to identify the type of renal cortical neoplasm in a biopsy sample. For each genomic region on urogenital array, the expected type (gain or loss) and frequency of genomic aberrations within the 4 subtypes of renal tumors are indicated. Multiple alterations have been found to have different occurrence rate in different subtypes and can be used to differentiate the subtypes.

TABLE 4
Genomic Regions on the Urogenital Cancer Array Associated with Renal Cortical Neoplasms

| | ccRCC* | | pRCC* | | chrRCC* | | Oncocytoma* | |
|---|---|---|---|---|---|---|---|---|
| Band | Gain | Loss | Gain | Loss | Gain | Loss | Gain | Loss |
| 1p36.32-36.33 | | 15-27% | | 11-67% | | 98-100% | | 29-46% |
| 1p35.2-36.11 | | 10-27% | | 11-24% | | 95-100% | | 29-46% |
| 1p32.3-33 | | 27-30% | | 11-24% | | 95-100% | | 29-46% |
| 1p21.3-22.2 | | 27-30% | | 11-24% | | 98-100% | | 29-46% |
| 1q21.1-23.1 | 33-36% | 27% | 15% | 20% | | 95-100% | | 29-33% |
| 1q25.3-31.2 | 10-36% | 27% | 15% | 20% | | 95-100% | | 29-33% |
| 1q32.1-32.3 | 10-33% | 27% | | 20% | | 95-100% | | 29-33% |
| 1q41 | 8-33% | 27% | | 20% | | 95-100% | | 29-33% |
| 1q42.2 | 10% | 27% | | 20% | | 95-100% | | 29-36% |
| 2p25.1 | | 27% | 33% | | | 94-95% | | |
| 2p23.3-24.1 | | 27% | | | | 94-95% | | |
| 2p22.1-22.2 | | 27% | | | | 94-95% | | |
| 2q14.2-14.3 | 13% | 22-27% | | | | 94-95% | | |
| 2q22.1-22.3 | | 22-27% | 50% | | | 94-98% | | |
| 2q34-35 | 16-24% | 8-27% | | | | 94-95% | | |
| 2q37.3 | | 9-27% | | | | 94-95% | | |
| 3p25.1-26.1 | 31% | 20-97% | 23% | 14% | | 23% | | |
| 3p21.2-21.31 | | 20-86% | 23% | 14% | | 23% | | |
| 3p13-14.2 | | 17-80% | 23% | 14% | | 23% | 7% | |
| 3q13.33-21.2 | | 14-15% | 23-55% | | | 23% | | |
| 3q26.2-26.31 | | 14-15% | 23-55% | | | 23% | | |
| 3q26.32-26.33 | | 14-15% | 23-55% | | | 23% | | |
| 3q28-29 | 12% | 14-15% | 23-55% | | | 23% | | |
| 4p16.2-16.3 | | 10-13% | | 20% | | | | |
| 4p12-14 | | 10-13% | | 20% | | | | |
| 4q28.1-28.3 | 10% | 10-50% | | 36% | | | | |
| 4q34.1-35.2 | 10% | 10-50% | | | | | | |
| 5p15.33 | 10-33% | | | 33% | | 27% | | |
| 5p15.31-5p15.1 | 10-33% | | | 33% | | 27% | | |
| 5p13.3-p13.1 | 10-33% | | | 33% | | 27% | | |
| 5q11.2 | 10-20% | | | | | 27% | | |
| 5q14.1-14.3 | 17-40% | | 12% | | | 27% | | |
| 5q21.2-22.1 | 17-40% | 33-38% | 12% | | | 27% | | |
| 5q23.1-23.2 | 15-100% | | 12% | | | 27% | | |
| 5q35.1-35.3 | 15-100% | | 12% | | | 27% | | |
| 6p23-25.2 | 11% | 10-33% | | 33% | | 88-91% | | |
| 6p22.3 | 11% | 10-33% | | | | 88-91% | | |
| 6p21.31-22.1 | 11% | 10-33% | 4% | | | 88% | | |
| 6q14.2-16.1 | | 15-33% | | 15-40% | | 88% | | |
| 6q16.3-21 | | 15-33% | | 15-40% | | 88% | | |
| 6q24.3-25.3 | | 25-33% | | 15-40% | | 88% | | |
| 7p22.3-21.3 | 12-33% | | 33-88% | | | | | |
| 7p21.2-21.3 | 12-33% | | 42-88% | | | | | |
| 7p14.3-15.2 | 12-33% | | 33-88% | | | | | |
| 7p11.2-12.1 | 12-33% | | 33-88% | | | | | |
| 7q11.22-21.11 | 22-53% | | 44-88% | | | | | |
| 7q31.31-31.2 | 10% | | 42-100% | | | | | |
| 7q36.1-36.2 | 10-32% | | 44-88% | | | | | |
| 8p23.2-23.3 | | 10-32% | | | | | | |
| 8p21.2-22 | | 15-32% | | | | | | |
| 8q13.3-21.13 | | | 25-55% | | | | | |
| 8q22.1-22.3 | | | 25-100% | | | | | |
| 8q24.13-24.21 | 10-74% | | 25-100% | | | | | |
| 9p24.2-p24.1 | | 14-47% | | | | | | |
| 9p21.2-p21.3 | | 29-89% | | 11-80% | | 40% | | |
| 9p13.2-21.1 | | 14-47% | | 11-36% | | 40% | | |
| 9q21.31-21.33 | | 10-26% | | | | 40% | | |
| 9q33.2-q33.3 | | 17-26% | | | | 40% | | |
| 10p13-15.3 | 7% | 10-27% | | | | 94-95% | | |
| 10q23.2-23.31 | 7% | 15-41% | 11% | | | 94-95% | | |
| 10q25.1-25.3 | 7% | 15-27% | 11% | | | 94-95% | | |
| 11q22.3-23.3 | | 10% | | | | | | |
| 12p12.3-13.31 | 11-24% | | 49% | | | | | |
| 12q13.2-14.1 | 17-74% | | 28-58% | | | | | |
| 12q24.21-24.31 | | | 33% | | | | | |
| 13q11-12.2 | | 12-27% | | 33-36% | | 85-94% | | |
| 13q13.3 | | 10-27% | | 33-36% | | 85-94% | | |
| 13q14.11-14.3 | | 10-27% | | 33-36% | | 85-94% | | |
| 13q21.2-22.1 | | 10-27% | | 33-36% | | 85-94% | | |
| 13q32.3-34 | | 10-27% | | 33-36% | | 85-94% | | |
| 14q23.1-23.3 | | 15-36% | | 15-20% | | | | 5-31% |
| 14q32.13-32.33 | | 15-45% | | 15-42% | | | | 5-31% |
| 16p13.12-13.3 | 15-20% | 10% | 39-40% | | | | | |
| 16p12.2-12.3 | 15-20% | 10% | 39-40% | | | | | |
| 16q21-23.3 | 15-32% | 8% | 32-55% | | | | | |
| 17p13.1-13.3 | 15-33% | 8-33% | | | | 76-93% | | |
| 17p11.2-12 | 15-33% | 10-28% | 47-97% | | | 76-90% | | |
| 17q12-21.31 | 11-33% | 27-28% | 47-97% | | | 76-90% | | |
| 17q25.1-25.3 | 11-33% | 6-28% | 47-97% | | | 76-90% | | |
| 18p11.31-11.32 | | 6-23% | | | | | | |
| 18q11.2 | | 13-63% | | 20% | | | | |
| 18q21.1-23 | | 13-63% | | 20% | | | | |
| 19p13.3 | 13-36% | | | 42% | | | | |
| 19q13.32-13.41 | 13-34% | 8-9% | | | | | | |
| 20p12.1-12.3 | 19-21% | | 30-40% | | | | | |
| 20q13.2-13.33 | 20-53% | | 30-55% | | | | | |
| 21q22.13-22.3 | 6-22% | 7% | | 14-50% | | 73-88% | | |
| 22q11.1-12.1 | 26% | 10-11% | | 42% | | | | |
| 22q13.2-13.32 | 22-53% | | 44-88% | | | | | |
| Xp11.22-11.23 | 10% | | 42-100% | | | | | |
| Xq13.2-13.3 | 10-32% | | 44-88% | | | | | |
| Xq21.33-22.1 | | 10-32% | | | | | | |
| Yp11.1-11.2 | | 15-32% | | | | | | |

*Gain and loss percentages represent the percentages of specimens found to show the CNA across studies. Clear cell RCC = ccRCC, papillary RCC = pRCC, chromophobe RCC = chrRCC.

The microarrays of the present invention may also be used to provide a prognostic purpose for kidney cancer, for one subtype, clear cell RCC (ccRCC). Table 5 lists the expected CNAs and associations with clinical and pathologic features associated with outcome.

TABLE 5
Genomic Aberrations in ccRCC Associated with
Pathologic and Clinical Markers of Outcome

| Band | Gain* | Loss* |
|---|---|---|
| 1p36.32-36.33 | | 15-27% (GH, S, M) |
| 1p35.2-36.11 | | 10-27% (PO, M, GH, S) |
| 1p32.3-33 | | 27-30% (GH, S, M) |
| 1p21.3-22.2 | | 27-30% (GH, S, M) |
| 1q21.1-23.1 | 33-36% (M) | 27% |
| 1q25.3-31.2 | 10-36% (M) | 27% |
| 1q32.1-32.3 | 10-33% | 27% |
| 1q41 | 8-33% (PO, GR, S) | 27% |
| 1q42.2 | 10% | 27% |
| 2p25.1 | | 27% |
| 2p23.3-24.1 | | 27% |
| 2p22.1-22.2 | | 27% |
| 2q14.2-14.3 | 13% | 22-27% (GH) |
| 2q22.1-22.3 | | 22-27% (GH) |
| 2q34-35 | 16-24% | 8-27% (GH) |
| 2q37.3 | | 9-27% (GH) |
| 3p25.1-26.1 | 31% | 20-97% (S, GL, GO) |
| 3p21.2-21.31 | | 20-86% (S, GL, GO) |
| 3p13-14.2 | | 17-80% (GL, S, GO) |
| 3q13.33-21.2 | | 14-15% |
| 3q26.2-26.31 | | 14-15% |
| 3q26.32-26.33 | | 14-15% |
| 3q28-29 | 12% (NON-VHL) | 14-15% |
| 4p16.2-16.3 | | 10-13% (PO, GL, S, C) |
| 4p12-14 | | 10-13% (PO, GL, S, C) |
| 4q28.1-28.3 | 10% | 10-50% (M, PO, GL, S) |
| 4q34.1-35.2 | 10% | 10-50% (M, GL, S) |
| 5p15.33 | 10-33% (GL) | |
| 5p15.31-5p15.1 | 10-33% (GL) | |
| 5p13.3-p13.1 | 10-33% (GL) | |
| 5q11.2 | 10-20% | |
| 5q14.1-14.3 | 17-40% | |
| 5q21.2-22.1 | 17-40% | 33-38% (GH, S, M) |
| 5q23.1-23.2 | 15-100% (GL, GO) | |
| 5q35.1-35.3 | 15-100% (GL, GO) | |
| 6p23-25.2 | 11% | 10-33% |
| 6p22.3 | 11% | 10-33% |
| 6p21.31-22.1 | 11% | 10-33% |
| 6q14.2-16.1 | | 15-33% |
| 6q16.3-21 | | 15-33% |
| 6q24.3-25.3 | | 25-33% |
| 7p22.3-21.3 | 12-33% (GH) | |
| 7p21.2-21.3 | 12-33% (GH) | |
| 7p14.3-15.2 | 12-33% (GH) | |
| 7p11.2-12.1 | 12-33% (GH) | |
| 7q11.22-21.11 | 22-53% | |
| 7q31.31-31.2 | 10% | |
| 7q36.1-36.2 | 10-32% | |
| 8p23.2-23.3 | | 10-32% |
| 8p21.2-22 | | 15-32% (GH) |
| 8p11.21-12 | | 15-27% (GH, S, M) |
| 8q13.3-21.13 | | |
| 8q22.1-22.3 | | |
| 8q24.13-24.21 | 10-74% | |
| 9p24.2-p24.1 | | 14-47% (GH, S, PO, C) |
| 9p21.2-p21.3 | | 29-89% (GH, S, PO, C) |
| 9p13.2-21.1 | | 14-47% (GH, S, PO, C) |
| 9q21.31-21.33 | | 10-26% (GH, S, PO, M) |
| 9q33.2-q33.3 | | 17-26% (GH, M) |
| 10p13-15.3 | 7% (GH) | 10-27% (M) |
| 10q23.2-23.31 | 7% (GH) | 15-41% (M) |
| 10q25.1-25.3 | 7% (GH) | 15-27% (M) |
| 11p15.3-15.4 | | |
| 11p12 | | |
| 11q13 | | |
| 11q14.1-14.2 | | |
| 11q22.3-23.3 | | 10% (GH, S, PO) |
| 12p12.3-13.31 | 11-24% | |
| 12q13.2-14.1 | 17-74% | |
| 12q24.21-24.31 | | |
| 13q11-12.2 | | 12-27% (S, GL, GO) |
| 13q13.3 | | 10-27% |
| 13q14.11-14.3 | | 10-27% |
| 13q21.2-22.1 | | 10-27% |
| 13q32.3-34 | | 10-27% |
| 14q11.2 | | |
| 14q23.1-23.3 | | 15-36% |
| 14q32.13-32.33 | | 15-45% (GL, S, PO) |
| 15q23-24.1 | | |
| 15q25.2-26.1 | | |
| 16p13.12-13.3 | 15-20% | 10% |
| 16p12.2-12.3 | 15-20% | 10% |
| 16q21-23.3 | 15-32% | 8% (GL) |
| 17p13.1-13.3 | 15-33% | 8-33% (GH, S, PO, M) |
| 17p11.2-12 | 15-33% | 10-28% (M, C) |
| 17q12-21.31 | 11-33% (GH, M) | 27-28% (M) |
| 17q25.1-25.3 | 11-33% (GH, M) | 6-28% (M) |
| 18p11.31-11.32 | | 6-23% (GH, S, PO, M) |
| 18q11.2 | | 13-63% (GH, S, PO, M) |
| 18q21.1-23 | | 13-63% (GH, S, PO, M) |
| 19p13.3 | 13-36% (M) | |
| 19q13.32-13.41 | 13-34% | 8-9% (GH, S, PO) |
| 20p12.1-12.3 | 19-21% | |
| 20q13.2-13.33 | 20-53% | |
| 21q22.13-22.3 | 6-22% (M) | 7% (S) |
| 22q11.1-12.1 | 26% (M) | 10-11% |
| 22q13.2-13.32 | 26% (M) | 10-11% |
| Xp11.22-11.23 | 6-10% | 10% |
| Xq13.2-13.3 | 8-28% (M) | 8-20% (GH, S, PO) |
| Xq21.33-22.1 | 10-28% (M) | 10-20% |
| Yp11.1-11.2 | | 15-55% (GO) |

*Gain and loss percentages represent the percentages of specimens found to show the CNA across studies.
GL = associated with low grade,
GH = associated with high grade,
S = associated with high stage/progression,
M = associated with metastasis/invasiveness,
C = associated with chemotherapy resistance/hormone refractory,
PO =associated with poor overall survival,
GO = associated with good overall survival,
Non-VHL = found only in sporadic and not VHL patients.

In another aspect, the invention can be used for the detection of CNAs associated with prostate cancer risk of progression, cancer recurrence after treatment and overall patient outcome. Table 6 lists the relevant regions on the urogenital array associated with these parameters in prostate cancer.

TABLE 6
Genomic Aberrations in Prostate Cancer Associated
with Pathologic and Clinical Markers of Outcome

| Band | Gain* | Loss* |
|---|---|---|
| 1p36.32-36.33 | 23-28% | 15-50% |
| 1p35.2-36.11 | | 15-50% (CR-M) |
| 1p32.3-33 | 28% | 15-39% |
| 1p21.3-22.2 | | 20-33% |
| 1q21.1-23.1 | 39-65% (CR-M) | |
| 1q25.3-31.2 | 50% (CR-M) | 18% |
| 1q32.1-32.3 | 7-45% (CR-M) | |
| 1q41 | 40% (CR-M) | |
| 1q42.2 | 18-30% (CR-M) | 40% |
| 2p25.1 | 33-60% | |
| 2p23.3-24.1 | 12-42% (M) | 25% |
| 2p22.1-22.2 | 12-45% | |
| 2q14.2-14.3 | | 25-44% |
| 2q22.1-22.3 | | 12-39% |
| 2q34-35 | 25% | |
| 2q37.3 | 54% (M) | 44% (CR-M) |
| 3p25.1-26.1 | | |
| 3p21.2-21.31 | 11-39% | 7-31% (CR-M) |
| 3p13-14.2 | 11-50% | 7-15% |
| 3q13.33-21.2 | 33% (M) | |
| 3q26.2-26.31 | 11-50% (M) | |
| 3q26.32-26.33 | 11-50% (M) | 27% |
| 3q28-29 | 20-40% | |
| 4p16.2-16.3 | | 20-44% (CR-M) |
| 4p12-14 | 25% | 32-40% (M) |
| 4q28.1-28.3 | 7-44% (M) | 8-44% |
| 4q34.1-35.2 | | 18-39% (CR-M) |
| 5p15.33 | 15-39% (PO) | |
| 5p15.31-5p15.1 | 15-39% (PO) | |
| 5p13.3-p13.1 | 15-39% (PO) | |
| 5q11.2 | 18-42% | |
| 5q14.1-14.3 | 9-33% | 14-33% (M) |
| 5q21.2-22.1 | 14% | 14-75% (M, GO) |
| 5q23.1-23.2 | 14-38% | 15-63% (M) |
| 5q35.1-35.3 | 18-33% (M) | 30% |
| 6p23-25.2 | | 25-30% (CR-M) |
| 6p22.3 | | |
| 6p21.31-22.1 | 13-23% | 28% |
| 6q14.2-16.1 | 25% | 14-60% (M, GO) |
| 6q16.3-21 | | 18-60% (M) |
| 6q24.3-25.3 | | |
| 7p22.3-21.3 | 23-70% (M) | |
| 7p21.2-21.3 | 18-51% (M) | |
| 7p14.3-15.2 | 18-42% (M) | |
| 7p11.2-12.1 | 18% (CR-M) | |
| 7q11.22-21.11 | 13-55% (M, PO) | |
| 7q31.31-31.2 | 20-50% (M, PO) | |
| 7q36.1-36.2 | 15-50% (C) | |
| 8p23.2-23.3 | | 18-100% (M, S) |
| 8p21.2-22 | | 10-75% (PO, M, S) |
| 8p11.21-12 | | 23-71% (M, PO) |
| 8q13.3-21.13 | 18-50% (M) | |
| 8q22.1-22.3 | | |
| 8q24.13-24.21 | 18-77% (M, GH) | |
| 9p24.2-p24.1 | | 33% |
| 9p21.2-p21.3 | | |
| 9p13.2-21.1 | 22-39% (C) | |
| 9q21.31-21.33 | | |
| 9q33.2-q33.3 | 6-36% (M) | 15% |
| 10p13-15.3 | 18% | 18-47% (CR-M) |
| 10q23.2-23.31 | | 12-90% (M, CR-M) |
| 10q25.1-25.3 | | 25-79% (M, CR-M) |
| 11p15.3-15.4 | 14-38% (M) | 22-28% |
| 11p12 | 14% | |
| 11q13 | 20-50% (PO) | |
| 11q14.1-14.2 | 22-75% (CR-M) | |
| 11q22.3-23.3 | 33% | |
| 12p12.3-13.31 | 6-25% | 6-30% |
| 12q13.2-14.1 | 56% | 27% |
| 12q24.21-24.31 | 28-67% | |
| 13q11-12.2 | | 39-50% (CR-M) |
| 13q13.3 | | 20-50% |
| 13q14.11-14.3 | | 18-88% (M, PO) |
| 13q21.2-22.1 | | 3-72% (PO, GH) |
| 13q32.3-34 | 20-60% | 29-59% (CR-M) |
| 14q11.2 | 22-36% | |
| 14q23.1-23.3 | 22-36% | |
| 14q32.13-32.33 | 14-33% | |
| 15q23-24.1 | | 7-22% |
| 15q25.2-26.1 | | 40% (CR-M) |
| 16p13.12-13.3 | 25-45% (M) | |
| 16p12.2-12.3 | 25-27% (M) | 24-25% |
| 16q21-23.3 | 27% | 18-82% (CR-M, M, GH) |
| 17p13.1-13.3 | | 20-61% (CR-M) |
| 17p11.2-12 | 10% | 20-30% (M) |
| 17q12-21.31 | 19-21% (M) | 20-45% |
| 17q25.1-25.3 | 10-58% (M) | 25-30% (CR-M) |
| 18p11.31-11.32 | 30% | |
| 18q11.2 | | 13-88% (M, CR-M, PO) |
| 18q21.1-23 | | 13-88% (M, CR-M, PO) |
| 19p13.3 | 25% | 20-30% |
| 19q13.32-13.41 | | 13-36% (CR-M) |
| 20p12.1-12.3 | 27-58% | 15-24% |
| 20q13.2-13.33 | 18-50% | 21-22% |
| 21q22.13-22.3 | 20-39% | 20-44% |
| 22q11.1-12.1 | | 16-65% (CR-M, S) |
| 22q13.2-13.32 | 23% | 15-46% (CR-M) |
| Xp11.22-11.23 | 27-39% | 22% |
| Xq13.2-13.3 | 27-55% | |
| Xq21.33-22.1 | 11-27% | 36% |
| Yp11.1-11.2 | | 44% |

*Gain and loss percentages represent the percentages of specimens found to show the CNA across studies.
GL = associated with low grade,
GH = associated with high grade,
S = associated with high stage/progression,
M = associated with metastasis/invasiveness,
C = associated with chemotherapy resistance/hormone refractory,
PO = associated with poor overall survival,
GO = associated with good overall survival.

In other aspects, the microarrays of the present invention can be utilized to predict outcome of patients with bladder cancer. In particular, it can be used to determine the need for patients undergo chemotherapy. CNAs associated with pathologic and clinical features that predict outcome and response to chemotherapy are listed in Table 7.

TABLE 7
Genomic Aberrations in Bladder Cancer Associated
with Pathologic and Clinical Markers of Outcome

| Band | Gain* | Loss* | Band | Gain* | Loss*8 |
|---|---|---|---|---|---|
| 1p36.32-36.33 | | 20% | 8q13.3-21.13 | 6-61% (SH) | |
| 1p35.2-36.11 | | 20% | 8q22.1-22.3 | | |
| 1p32.3-33 | 6-21% | 20% | 8q24.13-24.21 | 21-62% | |
| 1p21.3-22.2 | 12% | 20% | 9p24.2-p24.1 | | 14-75% |
| 1q21.1-23.1 | 10-54% (SH) | | 9p21.2-p21.3 | | 14-100% |
| 1q25.3-31.2 | 10-54% (SH) | | 9p13.2-21.1 | | 14-75% |
| 1q32.1-32.3 | 10-54% (SH) | | 9q21.31-21.33 | | 11-51% (GH, SH) |
| 1q41 | 10-54% (SH) | | 9q33.2-q33.3 | 6% | 11-67% (GH, SH) |
| 1q42.2 | 10-54% (SH) | | 10p13-15.3 | 4-29% (SH) | |
| 2p25.1 | 6-30% | 22% | 10q23.2-23.31 | | |
| 2p23.3-24.1 | 6-30% | 22% | 10q25.1-25.3 | | 17-39% (PO) |
| 2p22.1-22.2 | 6-50% | 22% | 11p15.3-15.4 | 24% | 10-55% (M) |
| 2q14.2-14.3 | 14% | 4-61% (GO, SH) | 11p12 | | 10-55% (M) |
| 2q22.1-22.3 | 14% | 4-61% (GO, SH) | 11q13 | 8-65% (GH, SH) | |
| 2q34-35 | 14% | 4-61% (GO, SH) | 11q14.1-14.2 | 6% | 13-25% (SH) |
| 2q37.3 | 14% | 4-61% (GO, SH) | 11q22.3-23.3 | 22-45% | 13-31% (SH, PO) |
| 3p25.1-26.1 | 8-18% (SH) | 10-67% (SH) | 12p12.3-13.31 | | |
| 3p21.2-21.31 | 6-29% | 20-67% (SH) | 12q13.2-14.1 | 4-60% | |
| 3p13-14.2 | 29% | 9-20% (SH) | 12q24.21-24.31 | 6-14% | |
| 3q13.33-21.2 | 6-43% (SH) | | 13q11-12.2 | 14-100% | 15-51% (GH, M) |
| 3q26.2-26.31 | 6-29% (SH) | | 13q13.3 | 14-100% | 15-31% (GH, M) |
| 3q26.32-26.33 | 6-29% (SH) | | 13q14.11-14.3 | 14-100% | 15-31% (GH, M) |
| 3q28-29 | 6-29% (SH) | | 13q21.2-22.1 | 14-100% | 15-31% (GH, M) |
| 4p16.2-16.3 | | 8-52% | 13q32.3-34 | 14-100% | 11-31% (GH, M) |
| 4p12-14 | | 13-53% (PO) | 14q11.2 | | |
| 4q28.1-28.3 | | 10-77% (TCC) | 14q23.1-23.3 | | |
| 4q34.1-35.2 | 10-25% | 24-57% (TCC) | 14q32.13-32.33 | | |
| 5p15.33 | 18-56% (PO, GH, SH) | | 15q23-24.1 | | 8-20% |
| 5p15.31-5p15.1 | 18-56% (PO, GH, SH) | | 15q25.2-26.1 | | 8-20% |
| 5p13.3-p13.1 | 18-56% (PO, GH, SH) | | 16p13.12-13.3 | 8% | |
| 5q11.2 | 21-38% (PO, SH) | | 16p12.2-12.3 | | |
| 5q14.1-14.3 | | 6-45% (GH, SH) | 16q21-23.3 | | 10-57% (SH) |
| 5q21.2-22.1 | | 6-50% (PO, SH) | 17p13.1-13.3 | 27-30% | 10-100% (GH, SH, PO) |
| 5q23.1-23.2 | | 12-54% (PO, SH, GH) | 17p11.2-12 | 27-30% | 10-100% (GH, SH, PO) |
| 5q35.1-35.3 | | 6-50% (GH, SH) | 17q12-21.31 | 4-58% (TCC) | 19% |
| 6p23-25.2 | 8-57% | | 17q25.1-25.3 | 4-58% (SH) | |
| 6p22.3 | 12-57% (SH, GH) | | 18p11.31-11.32 | 25% | 3-30% |
| 6p21.31-22.1 | 8-57% | | 18q11.2 | 31% | 7-65% (SH, PO) |
| 6q14.2-16.1 | | 50% (TCC) | 18q21.1-23 | 31% | 7-65% (SH, PO) |
| 6q16.3-21 | | 10-61% (TCC, SH, GH, PO) | 19p13.3 | 20-100% | |
| 6q24.3-25.3 | | 11-61% (TCC, SH, GH, PO) | 19q13.32-13.41 | 5-19% | |
| 7p22.3-21.3 | 16-44% (SH) | | 20p12.1-12.3 | 16-100% | 22% |
| 7p21.2-21.3 | | | 20q13.2-13.33 | 23-73% (SH) | |
| 7p14.3-15.2 | 16-44% (SH) | | 21q22.13-22.3 | | |
| 7p11.2-12.1 | | | 22q11.1-12.1 | 6% | 26% |
| 7q11.22-21.11 | 26-57% | | 22q13.2-13.32 | 6% | 26-33% |
| 7q31.31-31.2 | | | Xp11.22-11.23 | 14-25% (PO) | 11% |
| 7q36.1-36.2 | 4-26% | | Xq13.2-13.3 | 11-28% (SH, PO) | |
| 8p23.2-23.3 | | 15-65% (SH, GH) | Xq21.33-22.1 | | |
| 8p21.2-22 | | 20-65% (SH, GH) | Yp11.1-11.2 | | 21-49% |
| 8p11.21-12 | | 15-65% (SH, GH) | | | |

*Gain and loss percentages represent the percentages of specimens found to show the CNA across studies.
GH = associated with high grade,
SH = associated with high stage/progression,
M = associated with metastasis/invasiveness,
PO = associated with poor overall survival,
GO = associated with good overall survival,
TCC = associated with bladder transitional cell carcinoma.

A person skilled in the art using the present disclosure would be able to identify even further correlations between alterations at specific genomic regions and further types of cancers and thus could apply the presently described methods and devices in even further applications. Such further applications are intended to be encompassed by the present invention.

In one embodiment, a method for diagnosing the type or predicting the outcome of prostate, renal or bladder tumors present in a sample may comprise providing a microarray as otherwise described herein. As noted above, the present invention encompasses a number of different variations of microarrays and all such microarrays could be used in the methods of the present invention. Preferably, the microarray used in the method comprises genomic regions wherein alterations in such regions correlate to the disease feature and outcome of the prostate, renal, or bladder tumors being tested for or which are anticipated likely to be present in the sample being tested.

In further embodiments, the methods may comprise providing the sample with labeled genetic material therein. In carrying out the method of the invention, a sample for testing may be provided in a form wherein any genetic material present in the test sample already has been subjected to a labeling procedure to provide labels suitable for use according to the invention. In other embodiments, the method may comprise the actual step of labeling the genetic material present in the sample. Any method suitable for labeling of genetic material, such as DNA, may be used according to the invention. For example, the DNA could be digested with a suitable material, such as Rsa I and/or Alu I, and then appropriately labeled. In one embodiment, fluorescent labeling may be used (such as, for example, Cyanine 5-dUTP (Cy5) or Cyanine 3-dUTP (Cy3) using Klenow DNA polymerase).

In addition to the labeled test genetic material (i.e., the genetic material in the sample taken from a biopsy to be tested), labeled reference genetic material is used. Such reference material may include genetic material from confirmed normal healthy individuals.

The method of the invention further may comprise hybridizing the labeled genetic materials (test and reference) with the genomic regions arrayed on the substrate. Any hybridization method useful in the art could be used in hybridizing the genetic materials with the genomic regions. One method could encompass combining the labeled genetic materials (test and reference), human Cot-1, a blocking agent, and a hybridization buffer, and allowing the labeled genetic materials to hybridize with the genomic regions on the microarray for a sufficient time (e.g., about 24 hours) under acceptable conditions (e.g., a temperature of about 65° C.). Hybridization kits and techniques commercially available, such as the ones from Agilent Technologies, could be used.

The methods of the present invention can further comprise analyzing the hybridization pattern of the labeled genetic materials to the genomic regions. Such is useful to detect the presence of alterations in the genetic material from the sample relative to the reference. Analyzing methods useful according to the present invention can vary depending upon the type of labeling used. Preferably, analyzing can be carried out using equipment useful to evaluate hybridization patterns and identify regions on the microarray where alterations in the test sample occur.

The methods of the present invention further can comprise analyzing the hybridization pattern of the labeled genetic materials to the genomic regions. Such method is useful to detect the presence of alterations in the genetic material from the sample relative to the reference. Analyzing useful methods according to the present invention can vary depending upon the type of labeling used. Preferably, analysis can be carried out using equipment useful to evaluate hybridization patterns and identify regions on the microarray where alterations in the test sample occur.

The methods of the present invention also can include correlating any detected alterations to the type and outcome of prostate, kidney, and bladder cancer associated with the alteration. Tables 4-7 provided herein exemplifies several correlations of alterations at specific genomic regions to three types of urogenital cancers. In these tables, only those alterations that occur at frequencies higher than at least 5% in a minimum of two studies are listed or associated with a phenotype.

In other embodiments of the methods of the present invention, alterations in the genetic material from the sample can be detected using any method known in the art including, for example, any one or more of the technologies selected from the group consisting of karyotyping, FISH, Flow-FISH, SKY, chromosomal-CGH, array-CGH, SNP-array, PCR, and DNA sequencing. Any DNA sequencing methods known in the art can be used in the methods of the present invention including, but not limited to, next-generation or second generation sequencing technologies such as, for example, Massively Parallel Sequencing and the next-generation sequencing technologies as described in Egan et al. (2012) Am. J. of Bot. 99(2):175-185, herein incorporated by reference. The phrase “next-generation sequencing” or NGS refers to sequencing technologies having increased throughput as compared to traditional Sanger- and capillary electrophoresis-based approaches, for example, with the ability to generate hundreds of thousands of relatively small sequence reads at a time. Some examples of next generation sequencing techniques include, but are not limited to, sequencing by synthesis, sequencing by ligation, and sequencing by hybridization. In particular embodiments, the DNA fragment library is sequenced using the Illumina MiSeq system (Illumina, Inc., San Diego, Calif., USA), Illumina HiSeq 2000 system (Illumina, Inc., San Diego, Calif., USA), Illumina HiSeq 2500 system (Illumina, Inc., San Diego, Calif., USA), or the PacBio RS II system (Pacific Biosciences of California, Inc., Menlo Park, Calif., USA).

Next-generation sequencing of the genetic material will result in a collection of individual sequences corresponding to individual nucleic acids in the genetic material. As used herein, the term “read” refers to the sequence of a DNA fragment obtained after sequencing. In some embodiments, sequencing produces about 500,000, about 1 million, about 1.5 million, about 2 million, about 2.5 million, about 3 million, or about 5 million reads from the DNA sequence library. In certain embodiments, the reads are paired-end reads, wherein the DNA fragment is sequenced from both ends of the molecule. Depending on size of an individual nucleic acid, the paired-end reads can result in the full-length sequence of the individual nucleic acid whereby there is an overlapping region of sequence of the paired-end reads. Typically, however, the paired-end reads will not overlap and the sequence obtained for an individual nucleic acid will be less than full-length.

In some embodiments of the invention involving the use of next-generation sequencing, the sequencing information obtained from sequencing of the genetic material will be analyzed and assembled into the sequences of the individual nucleic acids in the subgroup of nucleic acids using computer software such as, for example, CLC Assembly Cell v. 4.2.0 (CLC bio, Cambridge, Mass., USA), Velvet (Birney, 2008, Genome Res. 18(5):821-29), ABySS Simpson et al., 2009, Genome Res. 19(6):1117-1123), Allpath LG (Gnerre et al., 2011, PNAS 108(4):1513-1518, MSR-CA (Zimin et al., 2013, Bioinformatics. 29(21):2669-2677), or MIRA (available on the worldwide web at sourceforge.net/projects/mira-assembler).

DNA sequencing of the genetic material from a sample can comprise sequencing the entire human genome or any portion thereof, particularly one or more of the genomic regions disclosed herein. For example, a portion of nucleic acids in a sample of genetic material can be selected by, for example, methods involving the use of a set of bait sequences designed to hybridize to the desired portion of nucleic acids in the sample of genetic material. Such methods comprise hybridizing in solution nucleic acids in the sample to form a hybridization mixture and then isolating from the hybridization mixture the nucleic acids that are hybridized to the bait sequences from nucleic acids that are not hybridized to the bait sequences. The use of such bait sequences to select and isolate a subgroup of nucleic acids from a group of nucleic acids has been previously described in U.S. Pat. App. Pub. No. 20100029498 and Gnirke et al. (2009, Nat. Biotechnol. 27(2): 182-189), both of which are herein incorporated by reference. In certain embodiments of the present invention, the desired portion of nucleic acids in the sample are selected using the MYbaits target enrichment system according the manufacturer's directions (Mycroarray, Ann Arbor, Mich., USA), the SureSelect target enrichment system (Agilent Technologies, Santa Clara, Calif., USA), the TruSelect exome enrichment system (Illumina, Inc., San Diego, Calif., USA), or the NimbleGen target enrichment system (Roche NimbleGen, Inc., Madison, Wis., USA) according the manufacturer's directions with bait sequences designed to hybridize to one or more of the genomic regions disclosed herein.

The article “a” and “an” are used herein to refer to one or more than one (i.e., to at least one) of the grammatical object of the article. By way of example, “an element” means one or more element.

The following examples are presented by way of illustration, and not by way of limitation.

### Example 1

### Development of a Microarray for Diagnosis and Prognosis of Urogenital Cancers

A literature survey was performed to facilitate development of tools (particularly the microarray—e.g., the urogenital cancer array, also known as the UroGenRA® urogenital cancer array as described herein) for the use in the diagnosis and prognosis of specific urogenital cancers including prostate, kidney, and bladder cancers. Initially, an evaluation of the literature was performed for molecular genetic and cytogenetic applications in the study of gynecological cancers. These studies utilized chromosomal CGH, FISH, array CGH (BAC and oligonucleotide), SNP-array, ROMA, and/or PCR-based assessment of single gene copy numbers. Across these studies vastly differing technologies were utilized and even within technologies, different platforms were used, making direct comparisons and extraction of data difficult. In addition, different cut-offs and algorithms were used for data analysis again complicating comparisons of results. Importantly, most of the studies examined very small datasets of clinical specimens and clinical associations either could not be drawn or would not even attempted by the investigators. These studies along with few with larger datasets, however often comprised patients that had been treated in different centers using different treatment plans and different criteria for response and outcome. Quite often, clinical associations across studies were not consistent. Overall, while the literature survey facilitated the development of the tool, the tool was uniquely designed for the application, taking into account technical, biological, and clinical information and considerations. Indications for inclusion of a region for the detection of a CNA, be it either gain or loss, on the urogenital array were carefully balanced and included but was not limited to: frequency in each cancer type to be assessed, diagnostic potential in each type, prognostic potential in each cancer type, predictive potential in each cancer type, potential to be associated with higher grade or lower grade associated with risk of each cancer type for progression, potential in each type to be associated with a clinical phenotype and/or biological phenotype or genotype, technology utilized in study, algorithm used for aberration detection in study, method of reporting aberrations, sensitivity of study, clinical robustness and size of dataset utilized in study, year of study, build of human genome utilized in study, variation in builds of human genome, variations in reporting bands and physical location of bands relative to the nucleotide sequence, physical location in the genome with respect to nucleotide position, chromosomal band, and chromosome, physical nucleotide location of copy number variants that normally occur within the human population, distribution of the regions across the chromosomal complement to ensure successful normalization, balance of regions within each type of cancer and across cancer types to ensure successful normalization, and size of regions so to have the highest likelihood to contain the target of the region but minimize overall size of region to permit a higher number of regions to be represented on the array with the potential to provide information relevant to the cancer type.

Table 3 lists the regions used in one embodiment of a microarray according to the present invention, the regions being identified according to cytogenetic band and physical location within the chromosome, according to the hg19 assembly (http://genome.ucsc.edu/).

Tables 4-7 list the genomic regions represented on the microarray and the expected alteration for each region and correlates this information to each type and subtype of prostate, kidney, and bladder cancers identifiable according to this embodiment of the array. Also shown in these tables are the alterations associated mostly with a clinical feature or a biologic feature. Thus, in specific embodiments, the urogenital cancer array can comprehensively represent 709 Mbp (approximately one-fourth of the human genome), targeting regions that are commonly gained/lost in these urogenital malignancies.

In one embodiment, the overall format of the oligonucleotide array was designed taking into account the following considerations: documented genomic regions of gain/loss, duplicity of probes on the array, resolution of probes for documented gain/loss, ease of performance of array hybridization, ease of analysis of data for a clinical laboratory, and economic viability as discussed below. Oligonucleotide arrays were designed through eARRAY (Agilent Technologies) utilizing the library of probes within eARRAY that map to the respective regions including probes that map to both exons and introns. Also included in the array design was a “backbone” probe set of approximately 3,100 probes (provided by Agilent) that cover the entire chromosomal complement at a resolution of approximately 1 Mbp excluding regions of known CNA. The gynecological cancer array was designed in the 4×44,000 (4×44K) format allowing the hybridization of four independent samples to each slide.

In general, DNAs were labeled and hybridized to the arrays on glass slides essentially as recommended by the manufacturer. Specifically, 1 μg of each test and reference DNAs were digested with Rsa I and Alu I and then differentially labeled with Cyanine 5-dUTP (Cy5) or Cyanine 3-dUTP (Cy3) using Klenow. Alternatively, test and reference DNAs could also be fragments to the desired size range by heat. Following removal of unincorporated nucleotides, the amounts of DNA and specific activities were determined. Prior to hybridization, equal amounts of the test and reference DNAs were mixed (range of 1.5-3.0 μg each), together with human Cot-1, a blocking agent, and hybridization mix.

The glass slide substrate (containing 4 arrays) was hybridized for 24-48 hours at 65° C. and then washed according to the manufacturer's recommendations with the inclusion of a wash in acetonitrile and a stabilizing and drying agent to minimize the ozone-induced degradation of the fluorophores, in particular Cy5. The slides were scanned using an Agilent scanner providing a scanned image (.tif) from which data was extracted using Feature Extraction (Agilent) (using the entire array unless otherwise stated). This software also provides data reporting the quality of hybridization (QC Metrics). Data are then further analyzed in Genomic Workbench (Agilent) for aberration detection, using the ADM2 algorithm. Such analyses give a read out of the genomic intervals (exact nucleotide boundaries at the start and end of each genomic gain/loss interval along the length of each chromosome) that statistically exhibit gain or loss in each specimen relative to the normal DNA, as well as the extent of the CNA. Intervals less than 250 kbp showing gain/loss were excluded due to consistent mapping to known CNA locations according to the Database of Genomic Variants (http://projects.tcag.ca/variation/) and to the observation that such gain and loss was very often common to multiple specimens at low significance. Intervals of gain and loss were then compared with the location of the regions represented on UGenRA to determine if all or part of the region is gained/lost.

The accuracy and precision (ability to accurately predict genomic gains and losses) of the urogenital cancer array was assessed using the following three RCC cancer cell lines (A498, A704, and ACHN), and one bladder cancer cell line (UM-UC-3). These cell lines were chosen since they are cell lines derived from cancers targeted for the urogenital cancer array, genomic copy number profiles as determined by SNP6-arrays (Affymetrix, comprising 946K copy number probes) are publicly available (http://www.sanger.ac.uk/cgi-bin/genetics/CGP/cghviewer/CghHome.cgi). Importantly they were also chosen because they displayed a diversity of ploidy (2-4), a range of the total number of CNAs per cell line (few to many), a variety of size of CNAs (hundreds of kbp up to whole chromosomes), and a variety of type of CNA (gains/losses, homozygous loss, and amplifications). The expected gain/loss call for each region for the SNP-6 arrays was determined relative to the most common whole copy number for the entire genome as available (see, the world-wide web at: sanger.ac.uk/cgi-bin/genetics/CGP/cghviewer/CghHome.cgi). This was compared with the observed CNA as detected by the urogenital array. Table 8 shows both the expected CNAs for each region (E) and the observed CNAs (O) for each region on the urogenital array for each cell line. For visualization purposes, black-filled boxes for any region indicate genomic gain when greater than 80% of the region was gained and grey boxes indicate genomic loss when greater than 80% of the region was gained. Black and grey ovals indicate when less than 80% of the region was gained/lost respectively. Hatch ovals indicate regions wherein both genomic gain and loss were expected/observed in less than 80% of the region.

TABLE 8
Expected and Observed Genomic Gains and Losses for Four Cancer Cell
Lines (Three Kidney and One Bladder) Using the Urogenital Cancer Array

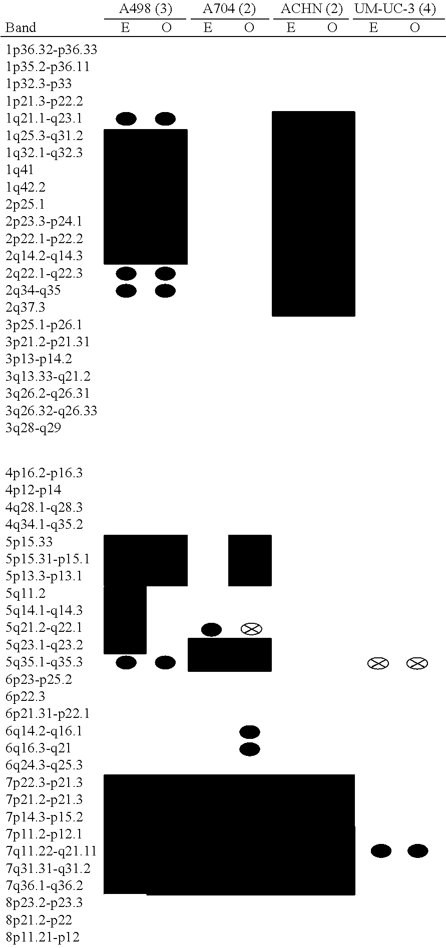 [SMILES: O ; O ; O ; O]

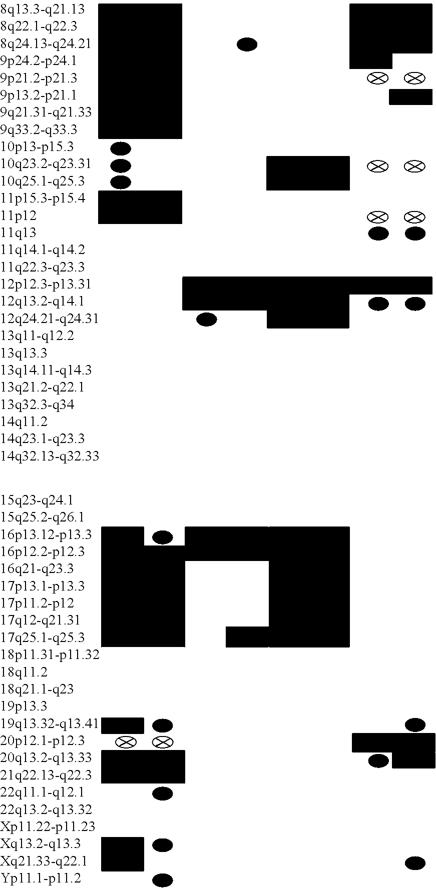

E = Expected, O = Observed, black box = gain of >80% of region, grey box = loss of >80% of region, hatched oval = gain and loss of <80% of region, black oval = gain of <80% of region, grey oval = loss of <80% of region.
Numbers in parentheses following each cell line name are the respective copy number used for determining gain/loss calls as derived from http://www.sanger.ac.uk/cgi-bin/genetics/CGP/cghviewer/CghHome.cgi

As expected, the cell lines showed a variety in the number of CNAs per cell line, the type of CNA (gain versus loss), and the size of the CNA. It was also expected given these are derived from epithelial cancers that in general they display a greater number of CNAs than other tumor types such as hematologic neoplasms. Lack of detection of the partial gains and losses (ovals) and/or observation of such alterations when not expected, can most likely be explained by an overlapping normal copy number variation at the site of the partial gain/loss (either in the cell line or matched by the reference DNA). While the average size for such normal copy variants is 250 kbp, there are others that can range up to 1-2 Mbps in size. CNAs are cataloged in the publicly available Database of Genomic Variants (http://projects.tcag.ca/variation). For the cell line, ACHN, overall good agreement between the expected and observed gains/losses was evident. The other three cell lines also showed good correlation, when considering “normalization” using a fraction versus whole copy number for calling gain/loss for the publicly available data. For UM-UC-3, then normalization based on a copy number between 3 and 4 (rather than 4), for A498 between 2 and 3 (rather than 3), and for A704 between 2 and 3 (rather than 2), yielded excellent correlation of gains/losses.

Importantly for the kidney cancer ccRCC cell lines (A498, A704, ACHN), many of the CNAs within regions represented on the urogenital array expected to be associated with kidney cancer, specifically ccRCC (Table 4 and 5) were detected. Similarly for the bladder cancer cell line (UM-UC-3), CNAs detected were frequently expected for bladder cancer as indicated by Table 7). These were quite distinct from the CNAs observed in the kidney cancer cell lines.

### Example 2

### A Preliminary Decision Tree for the Diagnosis of Renal Cortical Neoplasms

### Description of the Disease

In 2012, nearly 64,770 new cases of kidney cancer are estimated in United States and about 13,570 deaths from the disease are predicted to occur [1]. Renal cell carcinoma (RCC) is the most abundant form of kidney cancer, and despite being the most lethal can be cured by surgery if diagnosed at an early stage. RCC arises in the renal cortex and is the predominant malignant type of renal cortical neoplasm as shown in Table 9.

TABLE 9
Renal Cortical Neoplasm Histologic Subtype and Frequency

| Renal Cortical Neoplasm Histologic Subtype | Frequency |
|---|---|
| Benign | |
| Oncocytoma (OC) | 6-9% |
| Papillary adenoma | <1% |
| Metanephric adenoma | <1% |
| Nephrogenic adenofibroma | <1% |
| Malignant | |
| Clear cell (conventional) RCC (ccRCC) | 60-65% |
| Papillary RCC (pRCC) | 13-15% |
| Chromophobe RCC (chrRCC) | 6% |
| Collecting duct carcinoma | <1% |
| Medullary carcinoma | <1% |
| Tubulocystic RCC | <1% |
| RCC, unclassified | 7% |
| Mucinous tubular and spindle cell carcinoma | <1% |
| Translocation-associated carcinomas | <1% |
| Tumors of undetermined malignant potential | |
| Multilocular cystic RCC | <1% |

There are three main subtypes of RCC: clear cell (ccRCC) (comprises about 85% of RCC), papillary (pRCC), and chromophobe (chrRCC). These malignant subtypes (depending on other clinical features such as stage) are often treated with a combination of surgery/cryoablation, and targeted therapy including agents such as sunitinib and temsilorimus, the choice of which may depend on the RCC subtype. The next most frequent renal cortical neoplasm is oncocytoma (OC), which is benign and can be followed by active surveillance. It is important to note that by routine morphology, difficulties can arise in the differential diagnosis of chrRCC versus OC and even in specimens resected during surgery, up to 5-7% of RCCs are considered “unclassified” based on morphology alone. Thus, in the setting of RCC classification, there is a need to develop additional assays to assist routine morphologic classification of renal cortical neoplasm subtypes.

Renal neoplasms are often initially diagnosed as small renal masses (SRMs) by computerized tomography (CT) or magnetic resonance imaging (MRI), frequently when patients are asymptomatic. About 20% of SRMs are less than 2 cm and histologically benign, 55-60% are indolent and only about 20-25% are aggressive RCC [2, 3]. Current imaging techniques are of limited help in distinguishing whether a SRM is benign or malignant and in determining the RCC subtype. The available treatment options for SRMs include partial/radical nephrectomy, thermal ablation, and active surveillance, where one of the current medical challenges is to determine the optimal treatment for each patient. Despite advancement in diagnosis of renal tumors in recent years, the mortality rate of localized RCC (Stage I and II) is steadily increasing [4] probably due to unnecessary radical nephrectomies/overtreatment performed in early stage patients [5, 6]. Often patients with benign neoplasms are subjected to nephrectomy which may lead to unnecessary urologic complications [2]. This is particularly important given the median age of diagnosis of this disease between 60 and 70 years when patients often have other co-morbidities. Thus, accurate diagnosis of SRMs and renal neoplasms is highly desirable to make appropriate treatment decisions. Image-guided needle biopsies are emerging as popular diagnostic option for better understanding of tumor nature/subtype to aid in patient management [7]. Needle biopsies carry a minimal risk and current NCCN guidelines include needle biopsy as an option to confirm the malignancies of SRMs. However, a major challenge with needle biopsy is to obtain enough material for accurate diagnostication. Currently, about 15% of such needle biopsies are rendered non-diagnostic by routine histology [8]. Thus, there is a compelling need to develop ancillary assays that could assist histology to accurately classify SRMs.

Over the years, cytogenetic, molecular cytogenetic, cytogenomic, and molecular studies have shown that specific genetic alterations are associated with the four main renal cortical neoplasm subtypes. This is exemplified by inactivation of the Von Hippel-Lindau (VHL) gene (mapped to 3p25) in ccRCC, mostly evidenced as deletion of at least one allele. This alteration has been suggested to be the pathogenic event in this RCC subtype. Trisomy of chromosomes 7 and 17 are frequently observed in pRCC, and widespread monosomies in chrRCC. OC display a nearly diploid genome with loss of chromosome 1, 14 and Y [9-24]. In addition to copy number alterations, a subset of OC is characterized by chromosomal rearrangements at the CCND1 (11q13) locus [18, 25, 26]. Thus, based on genomic imbalance it is feasible to classify renal neoplasm subtypes to assist morphologic classification as has been suggested by several studies [14, 27-30].

In order to assist routine histology in the diagnosis of renal neoplasms both for needle biopsy specimens and fresh frozen resected specimens, the urogenital cancer array (UroGenRA) array-based comparative genomic hybridization (UroGenRA-RCC Array-CGH) assay has been developed based on differential genomic aberrations found in renal cortical neoplasms with potential diagnostic value.

### Regions to be Tested and Alterations

Table 3 lists the genomic regions represented on UroGenRA. The regions included in the array are based on genomic aberrations that have been well-documented in the literature by cytogenetic, molecular cytogenetic, cytogenomic, and molecular technologies to have potential diagnostic significance in renal cortical neoplasms [13, 14, 17, 23, 27-29, 31, 32]. Also represented in the array are additional regions for the purposes of array normalization and potential utilization of the array for gain/loss evaluation in other urogenital neoplasms such as prostate and bladder cancers. The regions to be assessed for gain and/or loss and used for classification are bolded.

Table 10 lists the aberrations that are observed in the four main renal cortical neoplasm subtypes that are used in UroGenRA-RCC array-CGH to classify the subtypes. For each aberration, the regions on UroGenRA that are utilized to assess the aberration are listed, along with the minimum criteria used to score positive for each aberration. When a target gene of the abnormality is known, as is the case for VHL, the region is localized to that gene and usually small. For other regions, where target genes are not well-documented or only suggested, larger regions are used.

Table 11 lists the aberrations detected by UroGenRA-RCC Array-CGH assay that are used for classification and the respective association of each aberration with one of the four subtypes. Also listed are the references supportive of the associations.

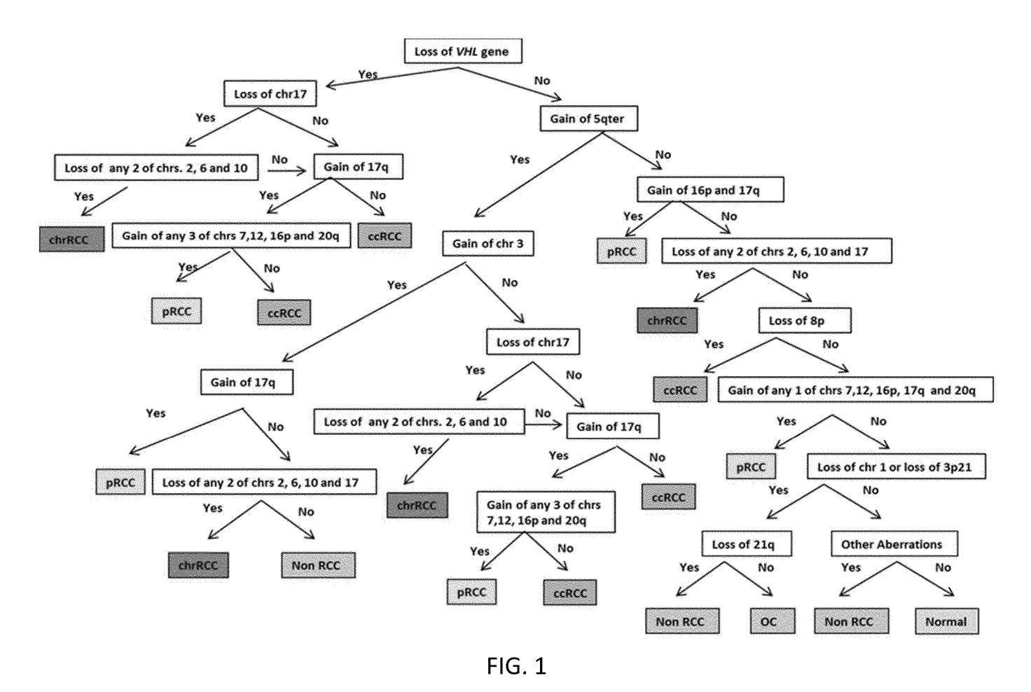

Detected aberrations within a specimen are used for classification according to an RCC-classification decision tree (FIG. 1) that was built and validated based on the following resources:

1) Published literature listed in Table 11.

2) Internal array-CGH study on core biopsy specimens.

3) Internal array-CGH study on over 100 fresh frozen RCC specimens (surgically-resected) and needle biopsy specimens.

4) Publicly available array-CGH log ratio profiles of 589 ccRCC, 75 pRCC and 65 chrRCC as part of ‘The Cancer Genome Atlas’ (TCGA) database (https://tcga-data.nci.nih.gov/tcga/tcgaHome2.jsp). The profiles were downloaded and analyzed in silico using Nexus Copy Number Analysis™ 6.1 (BioDiscovery Inc.) program.

Of note, specimens classified as “normal” (i.e. benign) by the decision tree are to be reflexed to FISH in order to rule in/out balanced CCND1 rearrangements which are characteristic of OC and would go undetected by array-CGH.

TABLE 10
Aberration and Criteria of UroGenRA ® Urogenital Cancer Array Regions

| Aberration | Criteria of UroGenRA ® Array Regions (Mbp) |
|---|---|
| Loss of VHL (chr3: 10.1-10.2 Mb) | 100% of chr3: 10.1-10.2 |
| Loss of chr2 | ≧80% of chr2: 9-12, 22-27, 37-39, 121-128, 138-145, 214-221, |
| | 238-243 |
| Gain of 17q12-q25 | ≧90% of chr17: 38-42, 74-78 |
| Gain of chr7 | ≧80% of chr7: 2.5-7.5, 10-14, 28-32, 53-57, 66-81, 115-119, 148-154 |
| Gain of chr12 | ≧80% of chr12: 10-15, 55-61, 116-123 |
| Gain of 16p13-p12 | ≧90% of chr16: 1-13, 21-24 |
| Gain of 20q13 | ≧90% of chr20: 55-62 |
| Gain of 5q (171-181 Mb) | ≧90% of chr5: 171-181 |
| Gain of chr3 | ≧80% of chr3: 8-15, 45-51, 60-72, 120-124, 168-172, 176-182, |
| | 190-196 |
| Loss of 6p25-q25 | ≧80% of chr6: 4-13.5, 17-23, 30-36, 84-94, 104-114, 149-161 |
| Loss of 10p15-q25 | ≧80% of chr10: 0-17, 88-92, 106-119 |
| Loss of chr17 | ≧80% of chr17: 0-9, 14-17, 38-42, 74-78 |
| Loss of 8p (1-5, 19-27 Mb) | ≧70% of chr8: 1-5, 19-27 |
| Loss of 1p36-q42 | >80% of chr1: 0-4, 26-31, 48-55, 92-95, 145-158, 181-192, 207-212, |
| | 217-221, 232-234 |
| Loss of 3p21.2-p21.31 | >90% of chr3: 45-51 |
| Loss of 21q22 | >90% of chr21: 38-47 |

TABLE 11
Aberrations Detected by UroGenRA-RCC Array-CGH Assay

| Aberration | Findings | References |
|---|---|---|
| Loss of VHL (chr3: 10.1-10.2 Mb) | Associated with ccRCC | [9-13, 17, 20, 21, 23, 32, 33] |
| Loss of chr2 | Associated with chrRCC | [9-11, 16, 17, 20, 31, 34] |
| Gain of 17q12-q25 | Associated with pRCC | [9-11, 17, 19, 20, 32, 35, 36] |
| Gain of chr7 | Associated with pRCC | [9-11, 17, 19, 20, 32, 35, 36] |
| Gain of chr12 | Associated with pRCC | [9-11, 17, 19, 20, 32, 35, 36] |
| Gain of 16p13-p12 | Associated with pRCC | [9-11, 17, 19, 20, 32, 35, 36] |
| Gain of 20q13 | Associated with pRCC | [9-11, 17, 19, 20, 32, 35, 36] |
| Gain of 5q (171-181 Mb) | Associated with ccRCC | [9-13, 17, 20, 21, 23, 32, 33] |
| Gain of chr3 | Associated with pRCC | [9-11, 17, 19, 20, 32, 35, 36] |
| Loss of 6p25-q25 | Associated with chrRCC | [9-11, 16, 17, 20, 31, 34] |
| Loss of 10p15-q25 | Associated with chrRCC | [9-11, 16, 17, 20, 31, 34] |
| Loss of chr17 | Associated with chrRCC | [9-11, 16, 17, 20, 31, 34] |
| Loss of 8p (1-5, 19-27 Mb) | Associated with ccRCC | [9-11, 16, 17, 20, 31, 34] |
| Loss of 1p36-q42 | Associated with OC | [9-11, 17, 20, 31, 37, 38] |
| Loss of 3p21.2-p21.31 | Associated with OC | Based on internal array-CGH study |
| Loss of 21q22 | Not associated with OC | Based on in silico data analysis |
| | | from TCGA database |

### Indications for Testing

1. Any RCC-suspected specimen (fresh frozen or needle biopsy tissue from primary kidney, benign or malignant tumor).
2. Any “RCC-Unclassified” specimen (that was not classifiable by histology alone) [39-42].

The UroGenRA-RCC Array-CGH assay is indicated as an ancillary assay to routine morphology that would assist in the histologic classification of renal masses either as core needle biopsies or resected specimens, both provided as fresh frozen tissue.

### REFERENCES

1. Siegel, R., D. Naishadham, and A. Jemal, Cancer statistics, 2012. CA Cancer J Clin, 2012. 62(1): p. 10-29.
2. Thomas, A. A. and S. C. Campbell, Small renal masses: toward more rational treatment. Cleve Clin J Med, 2011. 78(8): p. 539-47.
3. Frank, I., et al., Solid renal tumors: an analysis of pathological features related to tumor size. J Urol, 2003. 170(6 Pt 1): p. 2217-20.
4. Sun, M., et al., Age-adjusted incidence, mortality, and survival rates of stage-specific renal cell carcinoma in North America: a trend analysis. Eur Urol, 2011. 59(1): p. 135-41.
5. Zini, L., et al., Radical versus partial nephrectomy: effect on overall and noncancer mortality. Cancer, 2009. 115(7): p. 1465-71.
6. Thompson, R. H., et al., Radical nephrectomy for pT1 a renal masses may be associated with decreased overall survival compared with partial nephrectomy. J Urol, 2008. 179(2): p. 468-71; discussion 472-3.
7. Samplaski, M. K., et al., Renal mass sampling: an enlightened perspective. Int J Urol, 2011. 18(1): p. 5-19.
8. Reichelt, O., et al., Ultrasound-guided biopsy of homogenous solid renal masses. Eur Urol, 2007. 52(5): p. 1421-6.
9. Hagenkord, J. M., et al., Clinical genomics of renal epithelial tumors. Cancer Genet, 2011. 204(6): p. 285-97.
10. Moch, H., et al., Genetic aberrations detected by comparative genomic hybridization are associated with clinical outcome in renal cell carcinoma. Cancer Res, 1996. 56(1): p. 27-30.
11. Reutzel, D., et al., Genomic imbalances in 61 renal cancers from the proximal tubulus detected by comparative genomic hybridization. Cytogenet Cell Genet, 2001. 93(3-4): p. 221-7.
12. Klatte, T., et al., Cytogenetic profile predicts prognosis of patients with clear cell renal cell carcinoma. J Clin Oncol, 2009. 27(5): p. 746-53.
13. Arai, E., et al., Genetic clustering of clear cell renal cell carcinoma based on array-comparative genomic hybridization: its association with DNA methylation alteration and patient outcome. Clin Cancer Res, 2008. 14(17): p. 5531-9.
14. Barocas, D. A., et al., Renal cell carcinoma sub-typing by histopathology and fluorescence in situ hybridization on a needle-biopsy specimen. BJU Int, 2007. 99(2): p. 290-5.
15. Beleut, M., et al., Integrative genome-wide expression profiling identifies three distinct molecular subgroups of renal cell carcinoma with different patient outcome. BMC Cancer, 2012. 12(1): p. 310.
16. Bugert, P., et al., Specific genetic changes of diagnostic importance in chromophobe renal cell carcinomas. Lab Invest, 1997. 76(2): p. 203-8.
17. Chen, M., et al., Genome-wide profiling of chromosomal alterations in renal cell carcinoma using high-density single nucleotide polymorphism arrays. Int J Cancer, 2009. 125(10): p. 2342-8.
18. Jhang, J. S., et al., Renal oncocytomas with 11q13 rearrangements: cytogenetic, molecular, and immunohistochemical analysis of cyclin D1. Cancer Genet Cytogenet, 2004. 149(2): p. 114-9.
19. Jiang, F., et al., Chromosomal imbalances in papillary renal cell carcinoma: genetic differences between histological subtypes. Am J Pathol, 1998. 153(5): p. 1467-73.
20. Krill-Burger, J. M., et al., Renal cell neoplasms contain shared tumor type-specific copy number variations. Am J Pathol, 2012. 180(6): p. 2427-39.
21. Nagao, K., et al., Allelic loss of 3p25 associated with alterations of 5q22.3 approximately q23.2 may affect the prognosis of conventional renal cell carcinoma. Cancer Genet Cytogenet, 2005. 160(1): p. 43-8.
22. Pawlowski, R., et al., Loss of PBRM1 expression is associated with renal cell carcinoma progression. Int J Cancer, 2012.
23. Toma, M. I., et al., Loss of heterozygosity and copy number abnormality in clear cell renal cell carcinoma discovered by high-density affymetrix 10K single nucleotide polymorphism mapping array. Neoplasia, 2008. 10(7): p. 634-42.
24. Dondeti, V. R., et al., Integrative genomic analyses of sporadic clear cell renal cell carcinoma define disease subtypes and potential new therapeutic targets. Cancer Res, 2012. 72(1): p. 112-21.
25. Sukov, W.R., et al., CCND1 rearrangements and cyclin D1 overexpression in renal oncocytomas: frequency, clinicopathologic features, and utility in differentiation from chromophobe renal cell carcinoma. Hum Pathol, 2009. 40(9): p. 1296-303.
26. Zanssen, S., et al., Renal oncocytomas with rearrangements involving 11q13 contain breakpoints near CCND1. Cancer Genet Cytogenet, 2004. 149(2): p. 120-4.
27. Vieira, J., et al., Feasibility of differential diagnosis of kidney tumors by comparative genomic hybridization of fine needle aspiration biopsies. Genes Chromosomes Cancer, 2010. 49(10): p. 935-47.
28. Iqbal, M. A., et al., Use of FISH analysis for diagnosis of renal cell carcinoma subtypes. Ann Saudi Med, 1999. 19(6): p. 495-500.
29. Sanjmyatav, J., J. Schubert, and K. Junker, Comparative study of renal cell carcinoma by CGH, multicolor-FISH and conventional cytogenic banding analysis. Oncol Rep, 2005. 14(5): p. 1183-7.
30. Receveur, A. O., et al., Characterization of quantitative chromosomal abnormalities in renal cell carcinomas by interphase four-color fluorescence in situ hybridization. Cancer Genet Cytogenet, 2005. 158(2): p. 110-8.
31. Yusenko, M. V., et al., High-resolution DNA copy number and gene expression analyses distinguish chromophobe renal cell carcinomas and renal oncocytomas. BMC Cancer, 2009. 9: p. 152.
32. Matsuda, D., et al., Identification of copy number alterations and its association with pathological features in clear cell and papillary RCC. Cancer Lett, 2008. 272(2): p. 260-7.
33. Shuin, T., et al., Frequent somatic mutations and loss of heterozygosity of the von Hippel-Lindau tumor suppressor gene in primary human renal cell carcinomas. Cancer Res, 1994. 54(11): p. 2852-5.
34. Speicher, M. R., et al., Specific loss of chromosomes 1, 2, 6, 10, 13, 17, and 21 in chromophobe renal cell carcinomas revealed by comparative genomic hybridization. Am J Pathol, 1994. 145(2): p. 356-64.
35. Klatte, T., et al., Cytogenetic and molecular tumor profiling for type 1 and type 2 papillary renal cell carcinoma. Clin Cancer Res, 2009. 15(4): p. 1162-9.
36. Szponar, A., et al., Three genetic developmental stages of papillary renal cell tumors: duplication of chromosome 1q marks fatal progression. Int J Cancer, 2009. 124(9): p. 2071-6.
37. Junker, K., et al., Familial and sporadic renal oncocytomas—a comparative molecular-genetic analysis. Eur Urol, 2001. 40(3): p. 330-6.
38. Presti, J. C., Jr., et al., Comparative genomic hybridization for genetic analysis of renal oncocytomas. Genes Chromosomes Cancer, 1996. 17(4): p. 199-204.
39. Lopez-Beltran, A., et al., 2009 update on the classification of renal epithelial tumors in adults. Int J Urol, 2009. 16(5): p. 432-43.
40. Shen, S. S., et al., Recently described and emphasized entities of renal neoplasms. Arch Pathol Lab Med, 2007. 131(8): p. 1234-43.
41. Zisman, A., et al., Unclassified renal cell carcinoma: clinical features and prognostic impact of a new histological subtype. J Urol, 2002. 168(3): p. 950-5.
42. Karakiewicz, P. I., et al., Unclassified renal cell carcinoma: an analysis of 85 cases. BJU Int, 2007. 100(4): p. 802-8.

### Example 3

### Diagnostic Classification of Renal Tumors Using a Copy Number-Based Decision Tree Algorithm

### Abstract

Accurate diagnostic discrimination of major renal cortical neoplasms (clear cell, papillary, chromophobe and oncocytoma subtypes) is not only useful for predicting prognosis of the disease but also to plan appropriate treatment strategies which include active surveillance, cryoablation, partial and radical nephrectomy followed by with or without systemic therapy. Diagnosis solely by histopathology could often be challenging both in percutaneous needle biopsies as well as in surgically resected specimens. The aim of this study is to utilize the genomic imbalance associated with the major renal tumor subtypes to achieve correct diagnosis. Using TCGA (The Cancer Genome Atlas) copy number dataset, prior FISH study and published literature, a total of 15 genomic markers were identified. A decision tree algorithm was developed based on these genomic markers to assist in renal tumor subtyping. To validate the algorithm, genomic DNA extracted from 191 FFPE renal tumors were submitted to array-CGH (aCGH) using a custom array representing genomic regions commonly altered in urogenital neoplasms. Tumors evaluated included: 63 clear cell renal cell carcinoma (ccRCC), 57 papillary RCC (pRCC), 35 chromophobe RCC (chrRCC) and 36 oncocytoma (OC). Decision tree algorithm was applied on the aCGH data derived from the 191 tumors and aCGH-based classification was assigned in a blinded manner. Upon unblinding the histology diagnosis of these specimens, CCND1-rearrangement FISH was performed on OC specimens that were classified benign or not-classifiable by aCGH in order to obtain a definitive molecular diagnosis in these specimens. Correct molecular (FISH and aCGH combined) classification was obtained for 92% of ccRCC, 89% of pRCC, 97% of chrRCC and 78% of OC. Upon histological re-review of the slides for all the specimens, some of the misclassified specimens by aCGH had a mixed histology (mixed clear cell papillary RCC in the case of ccRCC/pRCC), low-level aberrations (gain of 17q in the case of OC) and a few others were not-classifiable (aberrations detected were not consistent with any subtype). Taken together in this validation study, a novel decision tree algorithm developed by us was able to correctly classify 89% of FFPE specimens belonging to the four major renal cortical neoplasms. The results presented in this study support the implementation of the decision tree algorithm for renal tumor classification in a clinical diagnostic setting.

### Introduction

Renal tumors are highly heterogeneous with several treatment modalities. About 48-66% of renal cancer is diagnosed asymptomatically as incidental renal masses detected by CT and MRI procedures [1]. Although surgical extirpation still remains the standard of treatment for renal tumors, surgery could be avoided in patients with benign or indolent neoplasms especially in older frail population who are poor surgical candidates. Also, about 25% of patients with renal masses who underwent nephrectomy were found to be affected by chronic kidney disease. In order to avoid unnecessary nephrectomies and associated co-morbidities, a variety of treatment options are becoming available to treat patients with renal tumors. The three major malignant renal cell carcinoma (RCC) subtypes namely clear cell RCC (ccRCC), papillary RCC (pRCC) and chromophobe RCC (chrRCC) are currently treated by nephrectomy or cryo-ablation with/without systemic therapy depending on the presence or absence of metastatic disease. Among the malignant subtypes, accurate molecular classification of the tumor is important in order to predict prognosis and to select appropriate systemic therapy to improve overall survival in these patients. On the other hand, benign neoplasms such as oncocytoma (OC) could be effectively managed by active surveillance without the need for surgery. A clear diagnostic discrimination between benign OC and malignant chrRCC is often difficult to achieve solely by histology due to their overlapping morphological features. In addition, a subset of RCC (about 6%) is categorized as ‘Unclassified’ by histology due to their complex morphology [2,3]. There is an increasing evidence that use of ancillary techniques such as molecular cytogenetic methods, gene expression profiling and immunohistochemistry could be beneficial in providing a more accurate diagnosis of renal tumors [4-9].

Renal neoplasms are characterized by specific genomic alterations which could be utilized for diagnostic and prognostic purposes. Some of the well-known copy number alterations (CNAs) associated with these subtypes are loss of Von Hippel-Lindau (VHL) gene (at 3p25, 10.1-10.2 Mb) in ccRCC, trisomy of chromosomes 7 and 17 in pRCC and widespread monosomies in chrRCC. OC display a nearly diploid genome with loss of chromosome 1, 14 and Y [6, 10-24]. In addition to CNAs, a subset of OC is characterized by chromosomal rearrangements at the CCND1 (11q13) locus [18, 25, 26]. Genomic alterations such as loss of 8p, 9p and 14q have been correlated with poor prognosis in RCC [27, 28]. Tumor grade, stage as well as histological subtype are crucial prognostic predictors in RCC. Thus, based on genomic imbalance it is feasible to classify renal neoplasm subtypes to assist morphologic classification as has been suggested by several studies [6-8, 29-31].

In this study, using TCGA (The Cancer Genome Atlas) copy number dataset, prior FISH study on renal tumors [32] and published literature, about 15 genomic regions were identified to serve as diagnostic markers for classifying the four major renal tumors—ccRCC, pRCC, chrRCC and OC. Using these genomic markers, a diagnostic algorithm was devised that could utilize the copy number data generated by array comparative genomic hybridization (aCGH) of renal tumors to provide subtype classification. The algorithm was validated using 191 surgically resected formalin fixed paraffin embedded (FFPE) specimens belonging to the above mentioned four renal tumor subtypes. Specimens that did not exhibit any CNAs were assigned benign by the decision tree. These benign specimens were evaluated for chromosomal rearrangement at the 11q13 (CCND1) locus by fluorescence in situ hybridization (FISH) assay to rule in/out oncocytoma. Some low-level aberrations detected by aCGH in a few oncocytoma specimens were also validated using FISH.

### Materials and Methods

### FFPE Specimens and DNA Extraction

A total of 191 surgically resected FFPE specimens were procured from Cleveland Clinic Foundation in an IRB-approved blinded study. 62 patients were female and 128 male; patients mean age was 61 years (range 27 to 88). The tumor burden in all the tissue specimens was around 80-90% as evaluated by H&E staining. The tissue sections were de-paraffinized overnight at 37° C. using sodium thiocyanate followed by proteinase K treatment at 60° C. overnight. After removing total RNA by RNase A treatment, genomic DNA (gDNA) was extracted using MasterPure DNA purification kit (Epicentre Biotechnologies, Chicago, Ill.). The quality (A₂₆₀/A₂₈₀) and quantity of gDNA was assessed by Nanodrop and all the gDNA were required to have A₂₆₀/A₂₈₀≧1.6 to be subjected to aCGH.
Targeted and Whole Genome aCGH

Specimens with high-molecular weight DNA (bulk of the DNA>800 bp in size) were subjected to heat fragmentation at 95° C. until the bulk of the DNA fragments reached 400-800 bp in size. Sex matched normal male and female gDNA (Promega, Madison, Wis.) were similarly heat-fragmented to serve as reference DNA. Test and reference DNA (1 μg) were differentially labeled with Cyanine 5- and Cyanine 3-dUTP respectively using random primers and Klenow fragment as recommended by the manufacturer (CGH Labeling Kit for Oligo Arrays, Enzo Lifesciences, Farmingdale, N.Y.). Unincorporated labeled nucleotides were removed by filtration (Ultracel—30K membrane filters, Millipore Corporation, Billerica, Mass.) and the DNA yield and labeling efficiency determined by Nanodrop. After labeling, a minimum of 2 μg output DNA was required to proceed to hybridization. Approximately equal amounts of labeled test and reference DNAs were combined and hybridized to a custom designed targeted oligonucleotide array in the 4×44K format (Agilent Technologies, Santa Clara, Calif.). The array comprised 17,348 features in duplicate representing 101 regions of the genome ranging in size from 2 to 28 Mb at an average resolution of 41.5 Kb, 301 features represented five times for reproducibility assessment, and 3,100 features in duplicate representing the entire genome at an average resolution of 1 Mb (Table 3). Hybridization was performed at 65° C. for 40 hours, washed according to manufacturer's recommendations followed by scanning on an Agilent DNA Microarray Scanner. Data were extracted using the Feature Extraction Software (version 10.7.3.1, Agilent). Raw data files have been deposited in GEO (accessible on the worldwide web at ncbi.nlm.nih.gov/geo). Nexus Copy Number 6.1 software (Biodiscovery Inc., Hawthorne, Calif.) was used to evaluate array hybridization quality (Q-score), log ratio profile visualization, and aberration detection. Analysis was performed using Rank Segmentation algorithm with duplicate probes combined, applied at significance of 5×10⁻¹⁰ and minimum probe length of eight. The Q scores which is a measure of array quality ranged from 0.02 to 0.35. Arrays were evaluated for CNAs with gain/loss at average log₂ ratio ±0.15. All genomic coordinates are according to the NCBI Build 37/hg19 assembly and known normal copy number variants (CNVs) were identified using the Database of Genome Variants (available on the worldwide web at projects.tcag.ca). Based on the genomic alterations detected, each specimen was assigned one of the four major renal tumor subtypes (ccRCC, pRCC, chrRCC, OC) in a blind manner according to the devised aCGH-based classification algorithm described below. About 14 representative specimens with low level CNAs (average log₂ ratio between ±0.15 and ±0.25) were re-submitted to whole genome 244K array (Agilent Technologies) to confirm the genomic changes. The aCGH methodology for 244K array was essentially the same as targeted array except that the hybridization was performed for 40 hrs instead of 24 hrs as recommended by the manufacturer.

### FISH Analysis

Four micron sections from OC specimens that were classified as benign and not-classifiable by aCGH were subjected to FISH using Paraffin Pretreatment Kit 1 (Abbott Molecular, Des Plaines, Ill.) according to the manufacturer's instructions. Following pretreatment, slides were denatured at 72° C. for five minutes and hybridized with CCND1 break-apart probes (to determine translocation at the 11q13 locus) or copy number probe 17q12 (to assess gain of 17q) at 37° C. overnight. Post-hybridization washes were carried out in 0.3% NP-40/2×SSC solution at 72° C. for two minutes. Slides were counterstained with DAPI and visualized and scored as described elsewhere [32]. A minimum of 200 nuclei were scored to assess CCND1 rearrangement. In specimens that were negative for rearrangement, up to 600 nuclei were scored to confirm negativity. The cut-off value for scoring cells as positive for rearrangement was derived from scores obtained two normal kidney FFPE specimens.

### Quantitative QPCR Analysis

Taqman-based QPCR copy number assays (Life Technologies, Foster City, Calif.) were performed using a StepOnePlus Real Time PCR System with copy-number assay primers/probes (mapping to KLHL11 and ABCA8) selected for two different loci in 17q arm. In brief, 5 ng DNA per well were amplified in duplicate per gene per DNA, using a locus at 13q (Hs03845777_cn) and RAG2 as control. The ΔΔCT method was calculated using the average of the control genes for two independent MF reference DNA dilutions and then averaged. Specimens with ratios ≧1.20 were considered positive for gain and ≦0.8, positive for loss.

### Results

### Analysis of TCGA Data to Identify Diagnostic Markers for Malignant RCC Subtypes

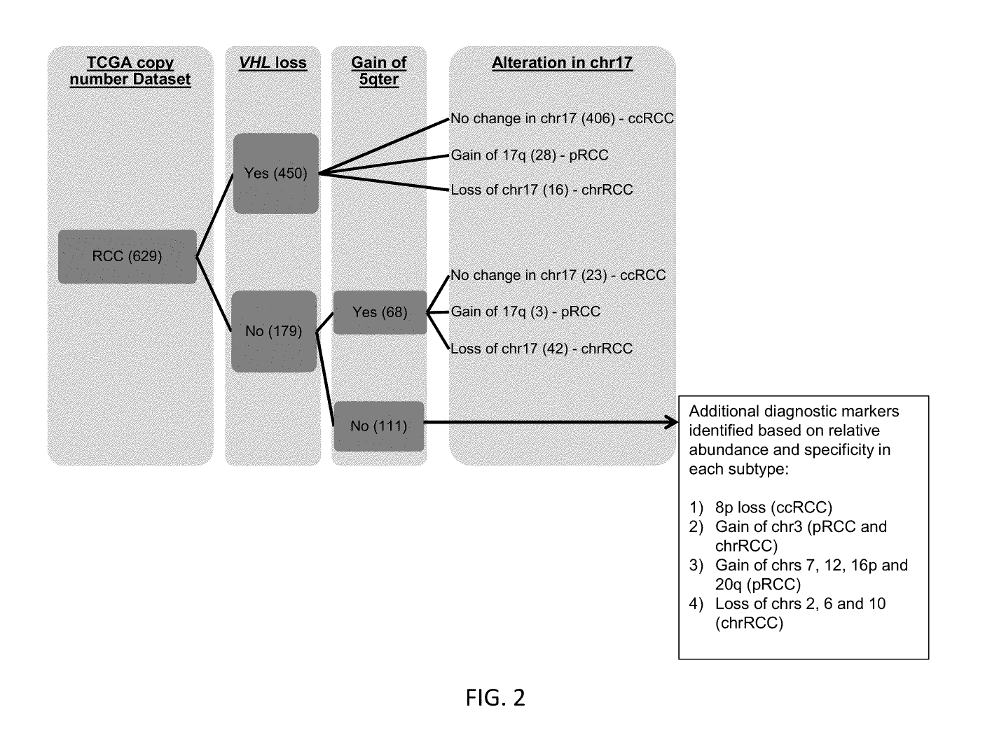

In order to develop an aCGH-based decision tree algorithm for classifying renal cortical neoplasms, publicly available whole genome copy number profiles from TCGA including a total of 629 malignant RCC specimens (489 ccRCC, 75 pRCC and 65 chrRCC) was considered. The level 3 copy number (segmented) data of all the 629 specimens were loaded in to Nexus 6.1 in a blinded manner. CNAs less than 300 Kb in size were filtered due to the abundance of CNVs in this size range. Segments with average log₂ ratio for gain/loss ≧±0.15 were considered as positive for a given CNA. Since ccRCC is the most predominant RCC subtype and is characterized by loss of VHL, the samples were initially categorized into two major groups depending on the presence or absence of VHL (3p25 locus, 10.1-10.2 Mb) loss. Among the 629 samples in the TCGA dataset, 450 exhibited VHL loss (FIG. 2). The remaining 179 samples were examined for gain of 5qter (169-181 Mb), the second most abundant and potentially specific region found in ccRCC. The next step was to subgroup the samples displaying VHL loss or 5qter gain (68/179) according to chr17 status; since chr17 loss was predominantly reported in chrRCC and gain of chr17 (more often 17q) was prevalent in pRCC. In the VHL loss group, 28 displayed gain of 17q while 16 exhibited loss of chr17. In samples with 5qter gain, three showed gain of 17q while 42 presented loss of chr17. Specimens with VHL loss or 5qter gain were preliminarily classified as ccRCC if was detected and chr17 was unaltered, as pRCC if gain of 17q was present and as chrRCC if loss of chr17 was noticed. Presence of VHL loss and 5qter gain guided initial classification of 82% (518/629) of TCGA dataset. Following preliminary subtyping of majority of TCGA samples, histology classifications of all the 629 samples were unblinded and additional diagnostic markers for each subtype were identified. Based on relative specificity and abundance in each subtype within the TCGA dataset the following markers were identified: Loss of 8p (1-27 Mb) for ccRCC; gain of chrs 7, 12, 16p and 20q for pRCC; loss of chrs 2, 6 and 10 for chrRCC subtype and gain of chr3 to distinguish ccRCC from the other two subtypes. Overall, published literature and the TCGA dataset provided 13 diagnostic markers (loss of VHL gene (3p25, 10.1-10.2 Mb), 8p (1-27 Mb), chrs 2, 6, 10 and 17; and gain of 5qter (169-181 Mb), chrs 3, 7, 12, 16p, 17q and 20q) for molecular diagnosis of the three major malignant RCC subtypes such as ccRCC, pRCC and chrRCC.

### Development of a Classification Algorithm for Subtyping Renal Tumors

Diagnostic markers for the benign OC subtype was derived from published literature (loss of chr1) and prior FISH study. (loss of 3p21) belonging to the four major renal tumor subtypes [32]. From prior FISH study using using 122 ex vivo core biopsies 10 OC specimens, loss of 3p21 locus without loss of VHL gene was suggestive of an OC subtype [32]. Taken together with the markers identified by TCGA dataset, a total of 15 diagnostic markers were obtained to classify above mentioned four major renal tumors. The criteria for calling these 15 CNAs used in the decision tree as positive (based on aCGH data) are provided in Table 12. According to the CNAs observed in the TCGA dataset, a preliminary tree was devised as shown in FIG. 2 and upon unblinding the TCGA histology data, the tree was further refined to achieve optimum sensitivity and specificity of subtype classification. Schematic representation of the final decision tree algorithm used to classify renal tumor specimens based on CNAs detected by aCGH is provided in FIG. 3. Loss of VHL (chr3:10.1-10.2 Mb) served as the primary node for stratifying specimens in to two major subgroups. Alteration in chr17 served as the secondary node for further classification. Depending on the presence or absence of additional markers, samples are assigned final classification as ccRCC/pRCC/chrRCC. In the VHL loss absent subgroup, gain of 5qter (171-181 Mb) served as a secondary node for further stratification. The next major marker used to triage specimens was gain of chr3, which pRCC and chrRCC from ccRCC subtype. One or more of additional pRCC/chrRCC markers is required to assign final classification in this subgroup. Specimens without VHL loss or 5qter gain were screened for the presence of 16p and 17q gain, the major markers for pRCC subtype. The next step was to complete the chrRCC diagnosis by testing samples for chrRCC markers. Samples were then analyzed for the final ccRCC marker, loss of 8p (1-27 Mb) for ccRCC classification. Due to overlapping occurrence of one or more of pRCC-related CNAs with OC marker—loss of chr1, pRCC diagnosis at this point involved straining samples that possessed one or more of pRCC markers without loss of chr1. Loss of chr1 or loss of 3p21 (45-51 Mb) served as the final node of the decision tree for OC subtype classification. If other aberrations are detected that are not consistent with any of the above-mentioned subtypes, then the specimen is categorized as not-classifiable. If no aberrations (excluding known normal copy number variants [CNVs]) are detected across the entire genome, then the specimen is classified as benign.

TABLE 12
List of the 15 genomic regions used by decision tree algorithm
and the criteria for calling each aberration as positive

| | Criteria (Mb) for Calling |
|---|---|
| Aberration | Aberration as Positive |
| Loss of VHL gene | 100% of chr3: 10.1-10.2 |
| Loss of chr 2 | ≧80% of chr2: 9-12, 22-27, 37-39, 121-128, |
| | 138-145, 214-221, 238-243 |
| Gain of 17q | ≧90% of chr17: 38-42, 74-78 |
| Gain of chr7 | ≧80% of chr7: 2.5-7.5, 10-14, 28-32, 53-57, |
| | 66-81, 115-119, 148-154 |
| Gain of chr12 | ≧80% of chr12: 10-15, 55-61, 116-123 |
| Gain of 16p | ≧90% of chr16: 1-13, 21-24 |
| Gain of 20q | ≧90% of chr20: 55-62 |
| Gain of 5q (171-181 Mb) | ≧90% of chr5: 171-181 |
| Gain of chr3 | ≧80% of chr3: 8-15, 45-51, 60-72, 120-124, |
| | 168-172, 176-182, 190-196 |
| Loss of chr6 | ≧80% of chr6: 4-13.5, 17-23, 30-36, |
| | 84-94, 104-114, 149-161 |
| Loss of chr10 | ≧80% of chr10: 0-17, 88-92, 106-119 |
| Loss of chr17 | ≧80% of chr17: 0-9, 14-17, 38-42, 74-78 |
| Loss of 8p (1-5, 19-27 Mb) | ≧70% of chr8: 1-5, 19-27 |
| Loss of chr1 (partial or | ≧15% of chr1: 0-4, 26-31, 48-55, 92-95, |
| entire) | 145-158, 181-192, 207-212, 217-221, |
| | 232-234 |
| Loss of 3p21.2-21.31 | ≧70% of chr3: 45-51 |

Authentication of CNAs Obtained by Targeted aCGH

Among the 191 specimens, four exhibited poor array quality and targeted aCGH was repeated for these four specimens using re-extracted DNA. Genomic aberrations detected by targeted aCGH for all 191 specimens were called positive or negative for the 15 diagnostic markers as per the criteria listed in Table 12 (data not shown). The targeted aCGH data obtained for the 15 CNA markers was scored for a given CNA with an average log₂ ratio between 0.15 and 0.25 is indicated as ‘Low’, above 0.25 is listed as ‘Yes’ and below 0.15 is tabulated as ‘No’. In order to verify low-level aberrations, gDNA from 14 representative specimens (carrying one or more of low-level CNAs) were submitted to whole genome 244K aCGH. These 14 specimens carried 34 low-level CNAs across the 15 genomic markers. 31/34 (91%) of these low-level aberrations could be detected by whole genome aCGH as well (data not shown) thus confirming most of the low-level aberrations. An exception to this occurred in the case of two specimens (CGI-015 and CGI-029), wherein low-level gain of 17q arm was detected as a sole abnormality. While whole genome aCGH could confirm low-level 17q gain in specimens which displayed other alterations in the genome, it could not confirm the same when occurring as a sole alteration. The two specimens (CGI-015 and CGI-029) were subjected FISH (using probe mapping to 17q12 locus) assay for further verification. Low-level 17q gain could not be validated by FISH as well. As a final approach to validate low-level whole arm 17q gain, the specimens were subjected QPCR using probes at 17q12 (KLHL11) and 17q24.2 (ABCA8) loci. QPCR could not confirm the whole arm 17q gain in these specimens suggesting that low-level 17q gain when detected as a sole aberration in a specimen should be considered as an array artifact. In the entire cohort, three specimens (CGI-006, -015 and -029) displayed low-level 17q gain as the sole abnormality.

### Validation of the Decision Tree for Malignant RCC Subtype Classification

Using the decision tree algorithm (shown in FIG. 3), an aCGH subtype classification (ccRCC, pRCC, chrRCC, OC, benign or not-classifiable) was assigned to each of 191 specimens in a blinded manner based on the presence or absence of the 15 genomic markers. Histology data was re-reviewed blindly by an independent pathologist (data not shown). Importantly, original and re-review histology of entire cohort was not revealed until all aCGH-based decision tree classification were performed.

Among the 191 specimens, original histology classified 63 as ccRCC, 57 as pRCC, 35 as chrRCC and 36 as OC. Re-review agreed with the histology of all except two specimens—1) CGI-064 which was chrRCC by original histology turned out to be benign upon re-review, and 2) CGI-110 was originally called pRCC, but re-review classified this specimen as ‘oncocytic with papillary features’. It is noteworthy that blinded decision tree classification of both these specimens was benign. For the purposes of calculating specificity and sensitivity of the assay, these two specimens were excluded leaving behind final analyzable cohort of 189 specimens comprising of 63-ccRCC, 56-pRCC, 34-chrRCC and 36-OC.

aCGH subtyping of ccRCC specimens: The decision tree algorithm (FIG. 3) was able to correctly classify 58 out of 63 ccRCC specimens yielding a sensitivity 90% for this subtype as shown in Table 13. Among the five mis-classified specimens, two were classified as pRCC, one benign, one not-classifiable and one OC. The benign specimen (CGI-120) exhibited no copy number change across the genome. The specimen (CGI-115) that was misclassified as OC displayed loss at 3p21 locus (45-51 Mb) without any change at the 3p25 (VHL, 10.1-10.2 Mb) locus. In prior FISH study using ex vivo needle biopsies of resected specimens, loss of 3p21 without loss of 3p25 was observed in 20% ( 2/10) OC specimens suggesting loss of 3p21 (without VHL loss) as a marker for OC [32]. This change was incorporated in the decision tree algorithm as a classifying aberration for OC subtype. Among the two specimens that were misclassified as pRCC (CGI-051 and CGI-190), re-review of histology indicated ccRCC with necrosis and rhabdoid features. The not-classifiable specimen (CGI-137) turned out be ‘clear cell papillary RCC’ upon histology re-review. The specificity of detection of ccRCC by aCGH assay was 97%.

TABLE 13
Diagnostic accuracy (sensitivity) and specificity of molecular
diagnosis (aCGH + CCND1 rearrangement FISH)
using histology as the gold standard

| Subtype | Sensitivity (%) | Specificity (%) |
|---|---|---|
| ccRCC | 58/63 (92) | 122/126 (97) |
| pRCC | 51/56 (91) | 129/133 (97) |
| chrRCC | 33/34 (97) | 155/155 (100) |
| OC | 27/36 (75) | 152/153 (99) |

aCGH subtyping of pRCC specimens: One or more of the following 5 genomic markers such as gain of chr7, chr12, 16p, 17q and 20q was used by the decision tree algorithm (FIG. 3) for pRCC classification. 51 out of 56 specimens are detected as pRCC by aCGH resulting in a sensitivity of 91%. Among the five specimens misclassified by aCGH, two specimens (CGI-039 ad CGI-081) exhibited CNAs that were not established markers for this subtype. These two specimens were therefore assigned as not-classifiable since the diagnostic significance of these genomic changes was unknown. The remaining three specimens (CGI-092, CGI-129 and CGI-150) had markers for both ccRCC and pRCC subtype. Since ccRCC was the primary decision point in the algorithm, these specimens were misclassified as ccRCC. The algorithm classified pRCC specimens with a specificity of 97%.

aCGH subtyping of chrRCC specimens: Among 34 chrRCC specimens, decision tree was able to accurately diagnose the tumor subtype of 33 specimens leading a sensitivity of 97%. Absence of widespread monosomies and the presence of low level chr12 gain lead to the misclassification of one chrRCC specimen (CGI-037) as pRCC. This renal tumor subtype was diagnosed by the decision tree algorithm with a specificity of 100%. Overall, the decision tree (FIG. 3) performed efficiently in classifying the malignant RCC subtypes.

### Validation of the Decision Tree for Benign/OC Subtype Classification

Due to lack of availability of large datasets for OC subtype, the markers for OC detection were derived from limited published reports [7, 20, 33]. In the original tree that was developed with TCGA dataset (data not shown), final pRCC subtyping was performed before OC classification using the following criteria: gain of one of the pRCC markers (without considering chr1 status). Among 36 specimens belonging to the OC subtype, 12 exhibited loss of chr1 in the absence of genomic markers for the other subtypes and hence were classified as OC by the original tree yielding a poor sensitivity (12/36, 33%). Upon unblinding histology, it was found that five out of nine OC specimens that were misclassified as pRCC by original tree had loss of chr1 along with pRCC markers (CGI-007, -009, -014, -023 and -031). By extending the criteria for final pRCC detection as ‘gain of any one of chrs7, 12, 16p, 17q and 20q without loss of chr1’ all these five specimens could be correctly classified. This minor change was made to the original tree (FIG. 1) to build the revised decision tree shown in FIG. 3 which was able to correctly classify 17/36 (47%) OC specimens. Among the 19 misclassified specimens: two were assigned malignant RCC subtype (1-ccRCC and 1-pRCC) with one of them showing 3p loss including VHL; 14 were classified as benign as they exhibited a quite genome by aCGH and three were regarded as not-classifiable as they displayed aberrations other than the 15 CNAs used by the revised tree.

A subset of OC has been reported in the literature to possess rearrangement at the 11q13 (CCND1) locus, a genomic alteration that could not be assessed by aCGH [25, 26]. Upon unblinding of the histology, 17 OC specimens (14 benign and three not-classifiable) that were mis-classified by aCGH were evaluated for CCND1 rearrangement by FISH assay to determine if any of these benign specimens carry this OC-specific rearrangement. Indeed, 10 of 17 specimens were found to display this alteration by FISH assay. FIG. 5 shows a representative specimen showing CCND1 rearrangement.

Taken together, 27 (17 by aCGH and 10 by FISH) out of 36 specimens were correctly detected for this subtype yielding a sensitivity of 75%. The specificity for OC diagnosis by the tree was 99%. Overall, the decision tree algorithm was able to correctly assign tumor subtype for 169 of 189 specimens yielding an overall diagnostic accuracy of 89%.

### Discussion

Molecular assays are increasingly being used to obtain diagnostic and prognostic information for a variety of cancers. In renal cancer, diagnosis and classification of renal neoplasms is becoming complex with the emergence of novel tumor subtypes and increased detection of small renal masses wherein renal mass sampling often results in damaged and insufficient tissue for diagnosis by histology. In RCC, needle biopsy-based diagnosis has proven valuable in clinical decision making especially in patients who are poor surgical candidates. Currently about 15-25% of needle biopsies have been reported to be non-diagnostic by histology and repeated biopsies are performed in such cases to arrive at a definitive diagnosis [34]. Histology-based diagnosis is often cumbersome for some tumors (eg: ‘Unclassified’ RCC), wherein an absolute molecular diagnosis would be beneficial to devise appropriate treatment. A number of molecular methods including FISH, immunohistochemistry, gene expression profiling and miRNA profiling are currently being evaluated for use as ancillary assays in the realm of renal tumor diagnosis [4, 7, 9, 35]. In the current study, a novel copy number-based algorithm was developed (using published literature, TCGA dataset and prior FISH study) and assessed for subtype classification using a large cohort of 191 surgically resected FFPE specimens belonging to the four major renal tumor subtypes (ccRCC, pRCC, chrRCC and OC).

The CNAs obtained by targeted aCGH data were comparable with that from whole genome aCGH with exception of one. Low-level gain of 17q when occurred as a sole abnormality appeared to be an array artifact as it could be not be verified either by whole genome aCGH or by FISH. Hence the three specimens (CGI-006, -015 and -029) with low-level 17q gain as a sole CNA were regarded as benign by aCGH. Decision tree-based classification was performed for all the specimens in a blinded manner following which the histology data was unblinded and compared with aCGH results. Upon unblinding histology a minor change was introduced in the tree to increase the efficiency of OC detection owing to the fact the intricate genomic changes occurring in OC is largely unknown. Also, lack of extensive literature or in silico dataset on OC a relatively less abundant subtype rendered poor classification of this subtype. In general, the tree was found to be superior in detecting malignant RCC compared to the benign OC subtype. This is partly reflected by the fact that malignant subtypes exhibited more number of genomic alterations than OC thus facilitating their correct stratification. Inclusion of CCND1-break apart FISH assay significantly increased OC detection from 47% by aCGH alone to 75% upon combining with FISH. Overall, the tree was able to provide definitive classification of 89% of renal tumors used in this study.

Some of the shortcomings of the present study are that the algorithm is not designed to detect: rarer renal tumor subtypes such as angiomyolipoma, collecting duct carcinoma, Xp11-translocation RCC and low grade oncocytic neoplasms; mutations, epigenetic changes and chromosomal translocations other than 11q13 rearrangement. An independent study with larger cohort of these subtypes is required to validate the diagnosis of such rare neoplasms. In addition to copy number changes, tumors carry mutations and epigenetic changes inclusion of which could further enhance the sensitivity of diagnosis [36, 37]. With the advent of next generation sequencing assays, it is feasible to integrate copy number, mutation and methylation changes in a single assay to achieve a maximally improved diagnosis for renal cancer.

### REFERENCES

1. Volpe A, Panzarella T, Rendon R A, et al. The natural history of incidentally detected small renal masses. Cancer 2004; 100(4):738-45.
2. Karakiewicz P I, Hutterer G C, Trinh Q D, et al. Unclassified renal cell carcinoma: an analysis of 85 cases. BJU Int 2007; 100(4):802-8.
3. Lopez-Beltran A, Carrasco J C, Cheng L, et al. 2009 update on the classification of renal epithelial tumors in adults. Int J Urol: official journal of the Japanese Urological Association 2009; 16(5):432-43.
4. Furge K A, Lucas K A, Takahashi M, et al. Robust classification of renal cell carcinoma based on gene expression data and predicted cytogenetic profiles. Cancer Res 2004; 64(12):4117-21.
5. Kuroda N, Tanaka A, Ohe C, Nagashima Y. Recent advances of immunohistochemistry for diagnosis of renal tumors. Pathol Int 2013; 63(8):381-90.
6. Barocas D A, Mathew S, DelPizzo J J, et al. Renal cell carcinoma sub-typing by histopathology and fluorescence in situ hybridization on a needle-biopsy specimen. BJU Int 2007; 99(2):290-5.
7. Sanjmyatav J, Schubert J, Junker K. Comparative study of renal cell carcinoma by CGH, multicolor-FISH and conventional cytogenic banding analysis. Oncol Rep 2005; 14(5):1183-7.
8. Wilhelm M, Veltman J A, Olshen A B, et al. Array-based comparative genomic hybridization for the differential diagnosis of renal cell cancer. Cancer Res 2002; 62(4):957-60.
9. Spector Y, Fridman E, Rosenwald S, et al. Development and validation of a microRNA-based diagnostic assay for classification of renal cell carcinomas. Mol Oncol 2013; 7(3):732-8.
10. Hagenkord J M, Gatalica Z, Jonasch E, Monzon F A. Clinical genomics of renal epithelial tumors. Cancer genetics 2011; 204(6):285-97.
11. Moch H, Presti J C, Jr., Sauter G, et al. Genetic aberrations detected by comparative genomic hybridization are associated with clinical outcome in renal cell carcinoma. Cancer Res 1996; 56(1):27-30.
12. Reutzel D, Mende M, Naumann S, et al. Genomic imbalances in 61 renal cancers from the proximal tubulus detected by comparative genomic hybridization. Cytogenet Cell Genet 2001; 93(3-4):221-7.
13. Klatte T, Rao P N, de Martino M, et al. Cytogenetic profile predicts prognosis of patients with clear cell renal cell carcinoma. J Clinical Oncol 2009; 27(5):746-53.
14. Arai E, Ushijima S, Tsuda H, et al. Genetic clustering of clear cell renal cell carcinoma based on array-comparative genomic hybridization: its association with DNA methylation alteration and patient outcome. Clin Cancer Res 2008; 14(17):5531-9.
15. Beleut M, Zimmermann P, Baudis M, et al. Integrative genome-wide expression profiling identifies three distinct molecular subgroups of renal cell carcinoma with different patient outcome. BMC Cancer 2012; 12(1):310.
16. Bugert P, Gaul C, Weber K, et al. Specific genetic changes of diagnostic importance in chromophobe renal cell carcinomas. Lab Invest 1997; 76(2):203-8.
17. Chen M, Ye Y, Yang H, et al. Genome-wide profiling of chromosomal alterations in renal cell carcinoma using high-density single nucleotide polymorphism arrays. Int J Cancer 2009; 125(10):2342-8.
18. Jhang J S, Narayan G, Murty V V, Mansukhani M M. Renal oncocytomas with 11q13 rearrangements: cytogenetic, molecular, and immunohistochemical analysis of cyclin D1. Cancer Genet and Cytogenet 2004; 149(2):114-9.
19. Jiang F, Richter J, Schraml P, et al. Chromosomal imbalances in papillary renal cell carcinoma: genetic differences between histological subtypes. Am J Pathol 1998; 153(5):1467-73.
20. Krill-Burger J M, Lyons M A, Kelly L A, et al. Renal cell neoplasms contain shared tumor type-specific copy number variations. Am J Pathol 2012; 180(6):2427-39.
21. Nagao K, Yamaguchi S, Matsuyama H, et al. Allelic loss of 3p25 associated with alterations of 5q22.3 approximately q23.2 may affect the prognosis of conventional renal cell carcinoma. Cancer Genet Cytogenet 2005; 160(1):43-8.
22. Pawlowski R, Muhl S M, Sulser T, et al. Loss of PBRM1 expression is associated with renal cell carcinoma progression. Int J Cancer 2012.
23. Toma M I, Grosser M, Hen A, et al. Loss of heterozygosity and copy number abnormality in clear cell renal cell carcinoma discovered by high-density affymetrix 10K single nucleotide polymorphism mapping array. Neoplasia 2008; 10(7):634-42.
24. Dondeti V R, Wubbenhorst B, Lal P, et al. Integrative genomic analyses of sporadic clear cell renal cell carcinoma define disease subtypes and potential new therapeutic targets. Cancer Res 2012; 72(1):112-21.
25. Sukov W R, Ketterling R P, Lager D J, et al. CCND1 rearrangements and cyclin D1 overexpression in renal oncocytomas: frequency, clinicopathologic features, and utility in differentiation from chromophobe renal cell carcinoma. Human Pathol 2009; 40(9):1296-303.
26. Zanssen S, Gunawan B, Fuzesi L, Warburton D, Schon E A. Renal oncocytomas with rearrangements involving 11q13 contain breakpoints near CCND1. Cancer Genet Cytogenet 2004; 149(2):120-4.
27. Beroud C, Fournet J C, Jeanpierre C, et al. Correlations of allelic imbalance of chromosome 14 with adverse prognostic parameters in 148 renal cell carcinomas. Genes Chromosomes Cancer 1996; 17(4):215-24.
28. Schullerus D, Herbers J, Chudek J, Kanamaru H, Kovacs G. Loss of heterozygosity at chromosomes 8p, 9p, and 14q is associated with stage and grade of non-papillary renal cell carcinomas. J Pathol 1997; 183(2):151-5.
29. Vieira J, Henrique R, Ribeiro F R, et al. Feasibility of differential diagnosis of kidney tumors by comparative genomic hybridization of fine needle aspiration biopsies. Genes Chromosomes Cancer 2010; 49(10):935-47.
30. Iqbal M A, Akhtar M, Al Dayel F, Ulmer C, Paterson M C. Use of FISH analysis for diagnosis of renal cell carcinoma subtypes. Ann Saudi Med 1999; 19(6):495-500.
31. Receveur A O, Couturier J, Molinie V, et al. Characterization of quantitative chromosomal abnormalities in renal cell carcinomas by interphase four-color fluorescence in situ hybridization. Cancer Genet Cytogenet 2005; 158(2):110-8.
32. Gowrishankar B, Cahill L, Arndt A E, et al. Subtyping of renal cortical neoplasms in fine needle aspiration biopsies using a decision tree based on genomic alterations detected by fluorescence in situ hybridization. BJU Int 2014.
33. Yusenko M V, Kuiper R P, Boethe T, et al. High-resolution DNA copy number and gene expression analyses distinguish chromophobe renal cell carcinomas and renal oncocytomas. BMC Cancer 2009; 9:152.
34. Samplaski M K, Zhou M, Lane B R, Herts B, Campbell S C. Renal mass sampling: an enlightened perspective. Int J Urol 2011; 18(1):5-19.
35. Skinnider B F, Amin M B. An immunohistochemical approach to the differential diagnosis of renal tumors. Semin Diagn Pathol 2005; 22(1):51-68.
36. Creighton C J, et al. Comprehensive molecular characterization of clear cell renal cell carcinoma. Nature 2013; 499(7456):43-9.
37. Sato Y, Yoshizato T, Shiraishi Y, et al. Integrated molecular analysis of clear-cell renal cell carcinoma. Nature Genet 2013; 45(8):860-7.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method of diagnosing renal cortical neoplasms, the method comprising:
(a) analyzing the genetic material from a sample obtained from a human individual to determine the presence or absence of the chromosomal aberrations set forth in FIG. 3 and/or Table 12; and
(b) classifying the subtype of renal cortical neoplasm in the sample using the decision tree shown in FIG. 3.

2. The method of claim 1, wherein the presence or absence one or more of the chromosomal aberrations is determined using the criteria set forth in Table 12.

3. The method of claim 1, wherein step (a) comprises:
(i) providing a microarray, said microarray comprising a substrate with a plurality of nucleic acid molecules corresponding to distinct genomic regions arrayed thereon, wherein each of the nucleic acid molecules is individually capable of hybridizing to material present in said sample, and wherein the genomic regions arrayed on the substrate are regions wherein an alteration therein is correlated to the diagnosis or prognosis of prostate, renal, or bladder tumors.
(ii) labeling said genetic material or portion thereof; and
(iii) hybridizing the labeled genetic material or portion thereof with the genomic regions arrayed on the substrate.

4. The method of claim 3, wherein analyzing comprises targeted array comparative genomic hybridization (array CGH) or whole genome array CGH.

5. The method of claim 3, wherein said analyzing said genetic material comprises analyzing the hybridization pattern of said labeled genetic material to said genomic regions to detect the presence of alterations in said genetic material from said sample.

6. The method of claim 3, wherein at least one of the distinct genomic regions is selected from the group consisting of: 1p36.32-p36.33, 1p35.2-p36.11, 1p32.3-p33, 1p21.3-p22.2, 1q21.1-q23.1, 1q25.3-q31.2, 1q32.1-q32.3, 1q41, 1q42.2, 2p25.1, 2p23.3-p24.1, 2p22.1-p22.2, 2q14.2-q14.3, 2q22.1-q22.3, 2q34-q35, 2q37.3, 3p25.1-p26.1, 3p21.2-p21.31, 3p13-p14.2, 3q13.33-q21.2, 3q26.2-q26.31, 3q26.32-q26.33, 3q28-q29, 4p16.2-p16.3, 4p12-p14, 4q28.1-g28.3, 4q34.1-q35.2, 5p15.33, 5p15.31-p15.1, 5p13.3-p13.1, 5q11.2, 5q14.1-q14.3, 5q21.2-q22.1 5q23.1-q23.2, 5q35.1-q35.3, 6p23-p25.2, 6p22.3, 6p21.31-p22.1, 6q14.2-q16.1, 6q16.3q-21, 6q24.3-q25.3, 7p22.3-p21.3, 7p21.2-p21.3, 7p14.3-p15.2, 7p11.2-p12.1, 7q11.22-q21.11, 7q31.31-q31.2, 7q36.1-q36.2, 8p23.2-p23.3, 8p21.2-p22, 8p11.21-p12, 8q13.3-q21.13, 8q22.1-g22.3, 8q24.13-q24.21, 9p24.2-p24.1, 9p21.2-p21.3, 9p13.2-p21.1, 9q21.31-q21.33, 9q33.2-g33.3, 10p13-p15.3, 10q23.2-q23.31, 10q25.1-q25.3, 11p15.3-p15.4, 11p12, 11q13, 11q14.1-g14.2, 11q22.3-q23.3, 12p12.3-p13.31, 12q13.2-q14.1, 12q24.21-q24.31, 13q11-q12.2, 13q13.3, 13q14.11-q14.3, 13q21.2-q22.1, 13q32.3-q34, 14q11.2, 14q23.1-q23.3, 14q32.13-q32.33, 15q23-q24.1, 15q25.2-q26.1, 16p13.12-p13.3, 16p12.2-p12.3, 16q21-q23.3, 17p13.1-p13.3, 17p11.2-p12, 17q12-q21.31, 17q25.1-q25.3, 18p11.31-p11.32, 18q11.2, 18q21.1-q23, 19p13.3, 19q13.32-g13.41, 20p12.1-p12.3, 20q13.2-q13.33, 21q22.13-q22.3, 22q11.1-q12.1, 22q13.2-q13.32, Xp11.22-p11.23, Xq13.2-q13.3, Xq21.33-q22.1, and Yp11.1-p11.2.

7. The method of claim 6, wherein the plurality of nucleic acid molecules comprises nucleic acid molecules corresponding to at least 10, 11, 12, 13, 14, 15, 16, 17, 18, 19, 20, 25, 30, or all of the distinct genomic regions set forth in claim 6.

8. The method of claim 6, wherein the plurality of nucleic acid molecules essentially consists of nucleic acid molecules corresponding to 10, 11, 12, 13, 14, 15, 16, 17, 18, 19, 20, 25, 30 or all of the distinct genomic regions set forth in claim 6.

9. The method of claim 3, wherein the plurality of nucleic acid molecules comprises or essentially consists of nucleic acid molecules corresponding to the distinct genomic regions set forth in Table 3.

10. The method of claim 1, wherein the subtypes are selected from the group consisting of clear cell renal cell carcinoma (ccRCC), papillary renal cell carcinoma (pRCC), and chromophobe renal cell carcinoma (chrRCC), oncocytoma (OC), Not-classifiable neoplasm, and Benign.

11. The method of claim 1, wherein the subtypes are clear cell renal cell carcinoma (ccRCC), papillary renal cell carcinoma (pRCC), and chromophobe renal cell carcinoma (chrRCC), oncocytoma (OC), Not-classifiable neoplasm, and Benign.

12. The method of claim 10, wherein the aberrations comprise the loss of VHL gene, the loss of chr2, the gain of 17q, the gain of chr7, the gain of chr12, the gain of 16p, the gain of 20q, the gain of 5qter, the gain of chr3, the loss of chr6, the loss of chr10, the loss of chr17, the loss of 8p, the partial or entire loss of chr1, and the loss of 3p21.2-21.31.

13. The method of claim 1, wherein analyzing comprises array CGH, wherein the method further comprises analyzing the genetic material to determine then presence or absence of rearrangements at the CCND1 (11q13) locus, and wherein the presence of the CCND1 rearrangements is indicative of OC.

14. The method of claim 1, wherein analyzing comprises next-generation sequencing.

15. The method of claim 14, wherein the method further comprises analyzing the genetic material to determine then presence or absence of rearrangements at the CCND1 (11q13) locus, and wherein the presence of the CCND1 rearrangements is indicative of OC.

16. The method of claim 1, wherein the sample is from a human individual previously diagnosed as having a small renal mass.

17. The method of claim 16, wherein the sample comprises the small renal mass.

18. The method of claim 1, wherein the sample is selected from the group consisting of a frozen resected sample, a needle biopsy sample, and a fresh resected sample.

19. The method of claim 1, wherein a computer is used in analyzing the genetic material in step (a) or in classifying the subtype in step (b), or in both step (a) and step (b).

20. The method of claim 19, wherein the decision tree is embodied in computer software.

21. A kit comprising a microarray suitable for the detection of genomic aberrations and the decision tree set forth in FIG. 3, wherein the aberrations comprise the loss of VHL gene, the loss of chr2, the gain of 17q, the gain of chr7, the gain of chr12, the gain of 16p, the gain of 20q, the gain of 5qter, the gain of chr3, the loss of chr6, the loss of chr10, the loss of chr17, the loss of 8p, the partial or entire loss of chr1, and the loss of 3p21.2-21.31.

22. The kit of claim 21, wherein the decision tree is on printed material or in a computer-readable form.

23. The kit of claim 21, wherein the decision tree is embodied in computer software.

24. The kit of claim 21, further comprising the criteria set forth in Table 12 for determining the presence or absence of one or more of the chromosomal aberrations, wherein the criteria are set forth in printed material, in a computer-readable form, or embodied in computer software.

25. The kit of claim 21, further comprising instructions for using the kit to detect the genomic aberrations, wherein the instructions are set forth in printed material, in computer-readable form, or embodied in computer software.

26. The kit of claim 21, further comprising instructions for classifying the subtype of renal cortical neoplasm in a sample comprising genetic material from a human individual.

27. The kit of claim 26, wherein the subtypes are clear cell renal cell carcinoma (ccRCC), papillary renal cell carcinoma (pRCC), and chromophobe renal cell carcinoma (chrRCC), oncocytoma (OC), Not-classifiable neoplasm, and Benign.

28. The kit of claim 21, wherein the microarray comprises a plurality of nucleic acid molecules corresponding to distinct genomic regions and at least one of the distinct genomic regions is selected from the group consisting of: 1p36.32-p36.33, 1p35.2-p36.11, 1p32.3-p33, 1p21.3-p22.2, 1q21.1-q23.1, 1q25.3-q31.2, 1q32.1-q32.3, 1q41, 1q42.2, 2p25.1, 2p23.3-p24.1, 2p22.1-p22.2, 2q14.2-q14.3, 2q22.1-q22.3, 2q34-q35, 2q37.3, 3p25.1-p26.1, 3p21.2-p21.31, 3p13-p14.2, 3q13.33-q21.2, 3q26.2-q26.31, 3q26.32-q26.33, 3q28-q29, 4p16.2-p16.3, 4p12-p14, 4q28.1-q28.3, 4q34.1-q35.2, 5p15.33, 5p15.31-p15.1, 5p13.3-p13.1, 5q11.2, 5q14.1-q14.3, 5q21.2-q22.1 5q23.1-q23.2, 5q35.1-q35.3, 6p23-p25.2, 6p22.3, 6p21.31-p22.1, 6q14.2-q16.1, 6q16.3q-21, 6q24.3-q25.3, 7p22.3-p21.3, 7p21.2-p21.3, 7p14.3-p15.2, 7p11.2-p12.1, 7q11.22-g21.11, 7q31.31-q31.2, 7q36.1-q36.2, 8p23.2-p23.3, 8p21.2-p22, 8p11.21-p12, 8q13.3-q21.13, 8q22.1-q22.3, 8q24.13-q24.21, 9p24.2-p24.1, 9p21.2-p21.3, 9p13.2-p21.1, 9q21.31-q21.33, 9q33.2-q33.3, 10p13-p15.3, 10q23.2-q23.31, 10q25.1-q25.3, 11p15.3-p15.4, 11p12, 11q13, 11q14.1-q14.2, 11q22.3-q23.3, 12p12.3-p13.31, 12q13.2-q14.1, 12q24.21-q24.31, 13q11-q12.2, 13q13.3, 13q14.11-q14.3, 13q21.2-q22.1, 13q32.3-q34, 14q11.2, 14q23.1-q23.3, 14q32.13-g32.33, 15q23-q24.1, 15q25.2-q26.1, 16p13.12-p13.3, 16p12.2-p12.3, 16q21-q23.3, 17p13.1-p13.3, 17p11.2-p12, 17q12-q21.31, 17q25.1-q25.3, 18p11.31-p11.32, 18q11.2, 18q21.1-q23, 19p13.3, 19q13.32-q13.41, 20p12.1-p12.3, 20q13.2-q13.33, 21q22.13-q22.3, 22q11.1-q12.1, 22q13.2-q13.32, Xp11.22-p11.23, Xq13.2-q13.3, Xq21.33-q22.1, and Yp11.1-p11.2.

29. The kit of claim 28, wherein the plurality of nucleic acid molecules comprises nucleic acid molecules corresponding to at least 10, 11, 12, 13, 14, 15, 16, 17, 18, 19, 20, 25, 30 or all of the distinct genomic regions set forth in claim 26.

30. The kit of claim 28, wherein the plurality of nucleic acid molecules essentially consists of nucleic acid molecules corresponding to 10, 11, 12, 13, 14, 15, 16, 17, 18, 19, 20, 25, 30, or all of the distinct genomic regions set forth in claim 26.

31. The kit of claim 21, wherein the microarray comprises a plurality of nucleic acid molecules corresponding to distinct genomic regions, the plurality of nucleic acid molecules comprising or essentially consisting of nucleic acid molecules corresponding to the distinct genomic regions set forth in Table 3.

32. A microarray for determining the type or predicting the disease progression of prostate, renal, or bladder tumors present in a sample, said microarray comprising a substrate with a plurality of nucleic acid molecules corresponding to distinct genomic regions arrayed thereon, wherein an alteration in at least one of said distinct genomic regions is correlated to the diagnosis or prognosis of one or more types of tumors mentioned above, and wherein said at least one distinct genomic region is selected from the group consisting of the distinct genomic regions set forth in at least one of Tables 3-8.

33. The microarray of claim 32, wherein at least one of the distinct genomic regions is selected from the group consisting of: 1p36.32-p36.33, 1p35.2-p36.11, 1p32.3-p33, 1p21.3-p22.2, 1q21.1-q23.1, 1q25.3-q31.2, 1q32.1-q32.3, 1q41, 1q42.2, 2p25.1, 2p23.3-p24.1, 2p22.1-p22.2, 2q14.2-q14.3, 2q22.1-q22.3, 2q34-q35, 2q37.3, 3p25.1-p26.1, 3p21.2-p21.31, 3p13-p14.2, 3q13.33-q21.2, 3q26.2-q26.31, 3q26.32-q26.33, 3q28-q29, 4p16.2-p16.3, 4p12-p14, 4q28.1-g28.3, 4q34.1-q35.2, 5p15.33, 5p15.31-p15.1, 5p13.3-p13.1, 5q11.2, 5q14.1-q14.3, 5q21.2-q22.1 5q23.1-q23.2, 5q35.1-q35.3, 6p23-p25.2, 6p22.3, 6p21.31-p22.1, 6q14.2-q16.1, 6q16.3q-21, 6q24.3-q25.3, 7p22.3-p21.3, 7p21.2-p21.3, 7p14.3-p15.2, 7p11.2-p12.1, 7q11.22-q21.11, 7q31.31-q31.2, 7q36.1-q36.2, 8p23.2-p23.3, 8p21.2-p22, 8p11.21-p12, 8q13.3-q21.13, 8q22.1-g22.3, 8q24.13-q24.21, 9p24.2-p24.1, 9p21.2-p21.3, 9p13.2-p21.1, 9q21.31-q21.33, 9q33.2-g33.3, 10p13-p15.3, 10q23.2-q23.31, 10q25.1-q25.3, 11p15.3-p15.4, 11p12, 11q13, 11q14.1-g14.2, 11q22.3-q23.3, 12p12.3-p13.31, 12q13.2-q14.1, 12q24.21-q24.31, 13q11-q12.2, 13q13.3, 13q14.11-q14.3, 13q21.2-q22.1, 13q32.3-q34, 14q11.2, 14q23.1-q23.3, 14q32.13-q32.33, 15q23-q24.1, 15q25.2-q26.1, 16p13.12-p13.3, 16p12.2-p12.3, 16q21-q23.3, 17p13.1-p13.3, 17p11.2-p12, 17q12-q21.31, 17q25.1-q25.3, 18p11.31-p11.32, 18q11.2, 18q21.1-q23, 19p13.3, 19q13.32-g13.41, 20p12.1-p12.3, 20q13.2-q13.33, 21q22.13-q22.3, 22q11.1-q12.1, 22q13.2-q13.32, Xp11.22-p11.23, Xq13.2-q13.3, Xq21.33-q22.1, and Yp11.1-p11.2.

34. The microarray of claim 33, wherein the plurality of nucleic acid molecules comprises nucleic acid molecules corresponding to at least 2, 3, 4, 5, 6, 7, 8, 9, 10, 11, 12, 13, 14, 15, 16, 17, 18, 19, 20, 25, 30, or all of the distinct genomic regions set forth in claim 32.

35. The microarray of claim 31, wherein the plurality of nucleic acid molecules comprises or essentially consists of nucleic acid molecules corresponding to the distinct genomic regions set forth in Table 3.
